(19) United States
(12) Patent Application Publication
(10) Pub. No.: US 2007/0142399 A1
(43) Pub. Date: Jun. 21, 2007

(54) Dual function drugs and uses thereof

(75) Inventors: Charles A. Altar, Garrett Park, MD (US); Malcolm G. Taylor, Abingdon (GB); Derek Hook, Woodbury, MN (US); Michael G. Palfreyman, Annapolis, MD (US)

Correspondence Address: DARBY & DARBY P.C., P. O. BOX 5257, NEW YORK, NY, 10150-5257, US

(73) Assignee: Psychiatric Genomics, Inc., Gaithersburg, MD (US)

(21) Appl. No.: 10/561,784

(22) PCT Filed: Jun. 21, 2004
(86) PCT No.: PCT/US04/19973

§ 371 Date: Dec. 19, 2005

Related U.S. Application Data
(60) Provisional application No. 60/480,036, filed on Jun. 19, 2003.

(51) Int. Cl. A61K 31/495; C07D 241/04

(52) U.S. Cl. 514/255.03; 544/392

(57) ABSTRACT

Dual action compounds are provided that are inhibitors of catechol-omethyltransferase (COMT) enzyme and are also partial agonists or antagonists of the D2 receptor, or agonists of the D1 receptor, or interact in these ways with both D1 and D2 receptors. Use of the compounds for treating neuropsychiatric disorders, particularly schizophrenia and mild cognitive impairment, is also described.

### CONTINUITY DATA

This application claims priority to U.S. provisional application 60/480,036 filed on Jun. 19, 2004

### FIELD OF THE INVENTION

The present invention relates to compositions and methods for treating neuropsychiatric disorders, such as schizophrenia and mild cognitive impairment. In particular, the invention relates to novel compositions, that are useful for treating both the positive and negative symptoms of schizophrenia and mild cognitive impairment.

### BACKGROUND OF THE INVENTION

Schizophrenia is a severe, life-long, idiopathic psychiatric disorder with a polygenic component. It is composed of severe thought disorders, termed psychoses that are characterized by illogical, delusional, or paranoid thoughts. Schizophrenia typically has its onset in early adulthood with remissions and exacerbations throughout life. The disorder afflicts approximately 1% of most populations. The signs and symptoms of schizophrenia usually begin in late adolescence or early adulthood and are manifested in a highly diverse and complex constellation of clinical presentations. These have been subdivided into two broad categories, positive and negative signs and symptoms.

The positive components are typically the first to draw attention to the disorder and constitute the more overt manifestations of psychosis. These include false perceptions including hallucinations (usually auditory), in which the patient's internal dialogue is perceived to originate from others or from inanimate sources such as radios or cell phones. Delusions, bizarre and often repetitive behavior patterns that are inappropriate to setting, and disorganized speech characterize other manifestations of schizophrenia.

The negative components are less spectacular, although more enduring, and, in many respects, more disabling of the characteristics of the disorder. These include alogia, anhedonia, avolution, blunted affect, disorganized thoughts and social withdrawal. Impaired cognitive function, including memory and attention defects, may also occur as part of the spectrum of schizophrenia, but also exist in otherwise normal individuals as a common result of aging.

The majority of drugs developed thus far for treating schizophrenia have been predominantly effective in ameliorating the positive symptoms of the disease. Examples of such drugs include haloperidol, chlorpromazine, clozapine thioridazine, olanzapine and risperidone. This list includes both the typical antipsychotics as well as the class of antipsychotics having reduced adverse side effects known as atypical antipsychotics. These drugs all share the commonality of acting as antagonists (or partial agonists) at the D2 receptor. Indeed, high striatal occupancy (70-80%) of D2 receptors at clinically effective doses is a hallmark of essentially all antipsychotic agents thus far discovered (Altar, C. A. et al., Burger's Medicinal Chemistry and Drug Discovery (2003) 6, In Press).

Dopamine signaling and metabolism have long been a central focus of attempts to explain the biochemistry of schizophrenia. This hypothesis originated with Carlsson and Lindquist (Carlsson, A. et al., Acta Pharmacol Toxicol (Copenh) (1963) 20, 140-4), who found that dopamine turnover as measured by HVA (homovanillic acid) levels was increased in laboratory animals following the administration of neuroleptic drugs. Subsequent research has supported the dopamine hypothesis, and is principally based on the psychotomimetic effects of drugs which augment doparnine function in the CNS, the psychotolytic effects of dopamine depleting drugs such as reserpine, the D₂ receptor antagonism of all antipsychotic drugs, and the increases in dopamine release and D₃ receptors in the schizophrenic brain.

The role of D2 partial agonism in the treatment of the positive symptoms of schizophrenia is well established as demonstrated by the clinical success of the (atypical) antipsychotic aripiprazole. This quinoline derivative was shown in receptor binding studies to have lower intrinsic activity than dopamine itself (Grunder, M. et al., Eur Arch Psychiatry Clin Neurosci (2002) 252, 51) at the D2 receptor and is believed to act as a modulator of the dopamine D2 receptor: Aripiprazole apparently reduces cellular D2 receptor signaling by blocking the receptor if it is overstimulated and stimulates the receptor when higher activity is needed. (Bandelow, B. et al., German J Psychiatry (2003) 6, 9-16) In multiple phase two and three double blind, placebo controlled studies (Kane, J. et al., Int J Neuropsychopharmacol (2000) 3, S124).

Unfortunately, neither the D2 partial agonist approach nor D2 blockade with more conventional silent D2 antagonists specifically address the negative symptoms of schizophrenia and would be unlikely to improve the symptoms of mild cognitive impairment, as D2 antagonists generally worsen cognitive functions. Negative symptoms characterize most schizophrenic patients to some extent, and constitute the major symptoms of about 15% of schizophrenics. The negative symptoms are, in many respects, more disabling of the characteristics of the disorder. Based on imaging studies in patients and the pharmacological responses to dopamine agonists, it is believed that the negative symptoms of schizophrenia result from deficiencies in D2 dopaminergic tone in the prefrontal cortex. Methods of treatment that effect the concentration and utilization of dopamine specifically in the prefrontal cortex would, therefore, likely have a beneficial effect in patients suffering the negative symptoms of schizophrenia.

Catechol-o-methyltransferase (COMT) is a widely distributed enzyme that catalyzes O-methylation of physiological substrates having a catechol structure in both the central and peripheral nervous systems. Substrates of COMT include adrenaline, noradrenaline, L-dopa and catechol estrogens. In the striatum and cortex-particularly important brain regions in understanding the etiology of schizophrenia—COMT degrades dopamine to the inactive metabolite 3-methoxytyramnine (3-MT) (Wood, P. L. et al., Pharmacol Rev (1988) 40, 163-87). Of these two brain regions, COMT apparently plays a more critical role in the regulation of synaptic dopamine levels in the frontal cortex. In this brain region, dopamine nerve terminals and the dopamine transporter (DAT) are relatively sparse, whereas COMT is highly expressed (Matsumoto, M. et al., Neuroscience (2003) 116, 127-37). In areas like the neostriatum, where dopamine nerve terminals and DAT are dense, COMT is sparse (Matsumoto, M. et al., Neuroscience (2003) 116, 127-37) and plays a relatively minor role in dopamine catabolism.

Electrophysiological studies in primates (Sawaguchi, T. et al., Science (1991) 251, 947-50, Williams, G. V. et al., Nature (1995) 376, 572-5) and rodents (Seamans, J. K. et al., J Neurosci (1998) 18, 1613-21), and neuroimaging studies in humans (Daniel, D. G. et al., J Neurosci (1991) 11, 1907-17; Mattay, V. S. et al., J Neurosci (1996) 16, 4816-22), have shown that dopamine plays an important role in modulating the activity of prefrontal circuitry during performance of working memory tasks. While there are many proteins involved in the biological actions of dopamine, catechol-O-methyltransferase (COMT), because it metabolizes released dopamine, may be an important factor during such prefrontally mediated tasks. Despite its widespread distribution in non-dopaminergic neurons and glia, pharmacological studies have shown that catabolic flux of synaptic dopamine through the COMT pathway is characteristic of the prefrontal cortex in contrast to the striatum (Karoum, F. et al., J Neurochem (1994) 63, 972-9). Studies of COMT knockout mice, similarly, have demonstrated that dopamine levels are increased only in the prefrontal cortex (Gogos, J. A. et al., Proc Natl Acad Sci USA (1998) 95, 9991-6) and that memory performance is enhanced (Kneavel, M. et al., Society for Neuroscience, New Orleans, (2000) 571.20 abstr). This regionally selective effect of COMT maybe because, in contrast to the striatum, dopamine transporters in the prefrontal cortex are expressed in low abundance and not within synapses (Lewis, D. A. et al., Adv Pharmacol (1998) 42,703-6; Sesack, S. R. et al., J Neurosci (1998) 18, 2697-708). As a consequence, dopamine released into the synapse appears to be inactivated by diffusion and ultimately degradation by COMT. These findings support the notion that variation in COMT activity may have neurobiological effects specific to the prefrontal cortex

The potential significance of COMT in the pathology of schizophrenia has been recently underscored by the finding that a valine allelic variant of the human enzyme is more thermally stable and thus at physiological conditions degrades dopamine at a faster rate than does the methionine-containing COMT allele. These allelic variants correlate with schizophrenic disease states. Egan and Weinberger have shown that a valine to methionine substitution in the gene that encodes for COMT results in a thermally less stable and biologically less active form of the enzyme (Egan, M. F. et al., Proc Natl Acad Sci USA (2001) 98, 6917-22). At body temperature, the homozygous methionine allele form of COMT has one-fourth of the activity of the valine allele (Lotta, T. et al., Biochemistry (1995) 34, 4202-10). This lessens the degradation of dopamine that normally occurs through the COMT pathway. Schizophrenics who inherit the valine-containing COMT allele, and degrade frontal cortex dopamine at a higher rate, show a poorer working memory and inefficiency of information processing in the prefrontal cortex than schizophrenics with the methionine allele (Egan, M. F. et al., Proc Natl Acad Sci USA (2001) 98, 6917-22; Joober, R. et al., Arch Gen Psychiatry (2002) 59, 662-3). Darvasi and colleagues (Shifman, S. et al., Am J Hum Genet (2002) 71, 1296-302) have also demonstrated a highly significant association by genetic linkage between schizophrenia and the valine-containing COMT allele. It is possible that this gene, which is on chromosome 22q11.

Current compounds known to inhibit COMT include the 5-nitrocatechol molecules entacapone and tolcapone. These compounds have been used to treat patients suffering from Parkinson's Disease as an adjunct therapy with L-dopa and (usually) dopa decarboxylase inhibitors. The utility of tolcapone and entacapone in Parkinson's disease derives from the ability for these compounds to inhibit the metabolism of L-dopa in the periphery, thereby increasing L-dopa concentrations in the plasma and increasing the amount ultimately reaching the brain. A unique difference between tolcapone and entacapone is the moderate ability of tolcapone to pass the blood brain barrier. This drug, hence, has the potential for acting centrally to inhibit COMT and provides a good starting point from which to rationally design drugs having an effect on the negative effects of schizophrenia associated with hypodopaminergia in the prefrontal cortex.

Decreased dopamine signaling mediated by the D1 receptor in the prefrontal cortex has also been identified as a potential cause for the negative symptoms of schizophrenia (Recently reviewed by Abi-Dargham, A. et al., Neuroscientist (2003) 9, 404-16). A number of D1 agonists have been approved for clinical use including feldopam, apomorphine and Abbot 431. Investigational agonists of the D1 receptor include SKF 83959, SKF 82598, CY 208-243 and dihhydrexine HCl. Decreased prefrontal D1 signaling has been in part attributed to a decreased presence of D1 receptors (see review). Whether this is a cause or result of the disease is incompletely clear, however, it is attractive to postulate that increasing signaling capability of the relatively fewer D1 receptors present in the prefrontal cortex would help to improve the negative symptoms associated with schizophrenia. A molecule capable of slowing metabolism of prefrontal cortex dopamine and increasing D1 signaling would be a particularly attractive candidate for the treatment the negative symptoms of schizophrenia

In addition to D1 and D2 dopaminergic signaling, it has long been suspected that receptors for serotonin (5-hydroxytryptamine; 5-HT) play a role in the pathology of schizophrenia. This theory had its beginnings in efforts to understand the observation that lysergic acid diethylamide (LSD) produces schizophrenic-like positive symptoms such as hallucinations. Initial investigation of this phenomenon indicated that LSD had partial agonist properties on central 5-HT2A receptors in the brain (Aghajanian, G. K., Annu Rev Pharmacol (1972) 12, 157-68; Berridge, M. J. et al., Br J Pharmacol (1974) 51, 269-78) and, therefore, the pathogenesis of schizophrenia was thought to result from excessive serotonin, stimulation of hallucinogenic receptors.

Interest in CNS serotinergic signaling as a target for treating schizophrenia has been kindled by the clinical success of clozapine, thioridazine and newer atypical antipsychotics. These compounds maintain partial D2 antagonism, however, more potently antagonize the 5HT-2A receptor than D2 receptors, in striatum and particularly in cortical brain regions (Altar, C. A. et al., Brain Res Bull (1986) 16, 517-25). Indeed, the 5HT-2A receptor has become increasingly implicated as an important effector in schizophrenia: Genetic analysis of schizophrenic individuals indicates that polymorphism in the gene (conservative substitution of thymidine for cytidine at amino acid 102) correlates with an increased risk for the disease (Williams, J. et al., Lancet (1996) 347, 1294-6). The ability of atypical antipsychotics to partially agonize 5-HT1A receptors, including aripiprazole (Jordan, S. et al., Eur J Pharmacol (2002) 441, 137-40), implicates this second serotonin receptor as a suitable target for the chimeric compounds described in this invention.

In summary, a novel treatment for the positive and negative symptoms of schizophrenia could be obtained with a compound that combines D2 partial agonism, or antagonism, with COMT inhibition. Such a compound would be expected to block D2 receptors in areas such as the neostriatum/nucleus accumbens, where dopamine tone is high and where COMT plays little role in dopamine signaling. A D2 partial agonist-COMT inhibitor is predicted to augment dopamine signaling in neocortical areas, where dopamine tone and innervation are quite sparse and where COMT plays a predominant role (FIG. 1). The D1 agonist-COMT inhibitor compound would augment DA signaling in the frontal cotex by its direct D1 agonism, and by augmenting dopamine in the synapse as a result of COMT antagonism. The beneficial effect of dopamine upon cognition by activation of the D1 receptors in the prefrontal cortex (Granon, S. et al., J Neurosci (2000) 20, 1208-15; Floresco, S. B. et al., Behav Neurosci (2001) 115, 93-49), together with an inhibition of COMT, is thus predicted to be beneficial for the negative symptoms of schizophrenia and for mild cognitive impairments resulting from decreases in frontal cortical dopamine function.

D1 agonism or D2 partial agonism and COMT inhibition are a realistic pair of pharmacological targets for a single chemical entity because (1) the natural biological target of both actions is dopamine, and (2) potent D1 agonists and D2 partial agonists and potent COMT inhibitors already exist. Structural optimization of the bifimctional compound revolves around known dopamine pharmacophores and around compounds that bind with high affinity to the D1 and/or D2 receptor and those that inhibit COMT. Target leads are those that confer low (10-20%) signaling at the D2 receptor and that inhibit COMT, or those that confer high (80-100%) signaling at the D1 receptor and that inhibit COMT. The blockade of cortical D2 receptors by a partial D2 agonist, or by a silent D2 antagonist, discovered by this approach may not be expected to compromise the effects of augmented synaptic dopamine concentrations in the frontal cortex. Partial amelioration of negative symptoms is frequently observed with potent D2 antagonists like haloperidol, possibly because they do not block D1/D5 receptors whose stimulation is required for optimal working memory function in the prefrontal cortex (Williams, G. V. et al., Nature (1995) 376, 572-5). Thus a compound with partial D2 partial agonist and COMT inhibition can activate the prefrontal cortex to overcome the negative symptoms of schizophrenia without initiating extrapyramidal symptoms through activation of the nigrostriatal dopamine pathways. As discussed above, a dopamine D1 agonist compound that also inhibits COMT is beneficial for the negative symptoms of schizophrenia without the detrimental effects that a full D2 agonist has on the psychotic features of the disease. An ideal compound contains all three activities: D1 agonist; D2 partial agonist; COMT inhibition.

### SUMMARY OF THE INVENTION

The present invention provides compounds and methods useful for the treatment of schizophrenia and mild cognitive impairment. Additionally the present invention provides compounds and methods useful for the treatment of Parkinson's Disease, Tourett's syndrome, depression, Alzheimer's disease, senile dementia, anxiety disorders, ischemic disease states, obsessive compulsive disorder, migraine, amyotrophic lateral sclerosis, epilepsy, eating disorders, premenstrual syndrome, attention deficit hyperactivity disorders, bipolar disorders, sexual dysfunction, and psychoses comprising administering to a patient in need of said treatment a pharmaceutical composition comprising compounds according to the present invention. In part, the methods described herein are based on the administration of rationally designed compounds having the capability of acting on a plurality of biologically relevant sites to the etiology of schizophrenia. The pharmacological functions selected for these compounds were chosen because of their ability to act on both the positive and negative side effects of schizophrenia. Specifically, the compounds disclosed herein have the property of inhibiting the enzyme catechol-O-methyltransferase COMT and: 1.) acting as a dopamine D1 receptor agonist, or 2.) acting as a dopamine D2 receptor partial agonist or antagonist, or 3.) acting as both a D1 agonist and D2 partial agonist or antagonist. Additional preferred activities of the compounds include partial agonism of the 5HT1A receptor and/or antagonism of the 5HT2A receptor.

Another embodiment of the present invention is to provide compounds of the Formula A

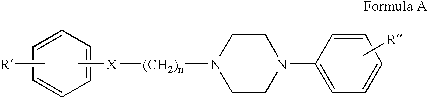

wherein R′ and R″ are independently selected for each position capable of substitution from the group consisting of halogen, hydroxyl, hydrogen, C₁-C₅ alkoxy, cyano (CN), and nitro (NO₂);

X is chosen from the group consisting of (C═O); O, NH, S,

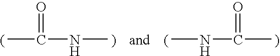

n is an integer from 1-6; and pharmaceutically acceptable salts thereof.

Another embodiment of the present invention is a compound of the Formula B 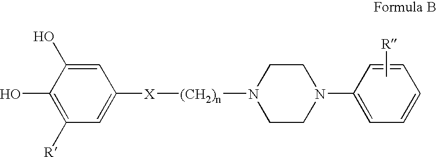
where R′, X, R″ and n are as previously defined for Formula A and pharmaceutically acceptable salts thereof.

Another embodiment of the present invention is a compound of the Formula C 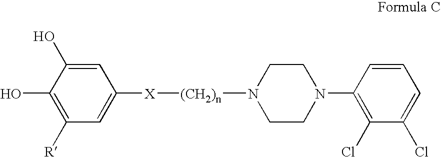
wherein R′, X and n are as defined for Formula A and pharmaceutically acceptable salts thereof.

A preferred R′ group for the preceding Formulas A, B and C is NO₂.

Preferred X groups for the preceding Formulas A, B and C are 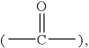
O and

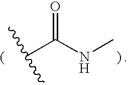

Further preferred for the preceding Formulas A, B and C is where n is 2, 3 or 4.

Another embodiment of the present invention is a compound of the Formula D

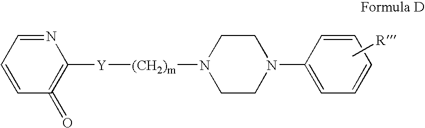

wherein R′″ is independently selected for each position capable of substitution or from the group consisting of halogen, hydroxyl, hydrogen, C₁-C₅ alkoxy, cyano (CN) and nitro (NO₂);

Y is chosen from the group consisting of 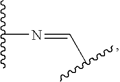
O, NH, S,

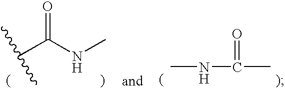

m is an integer from 1-6; and pharmaceutically acceptable salts thereof.

Further preferred is a compound of the Formula E 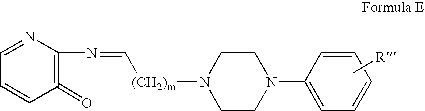
wherein R′″ and m are as defined for Formula D and pharmaceutically acceptable salts thereof.

Further preferred is a compound of the Formula F 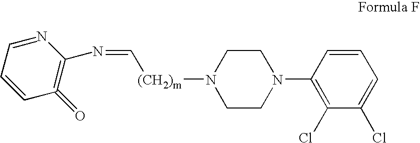
wherein m is as defined for Formula D and pharmaceutically acceptable salts thereof.

A further preferred embodiment is a compound of the Formula G 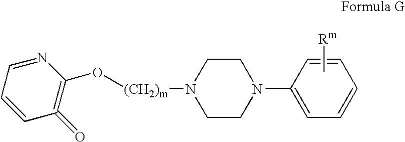
wherein R′″ and m are as previously defined in Formula D and pharmaceutically acceptable salts thereof.

A further preferred embodiment is a compound of the Formula H 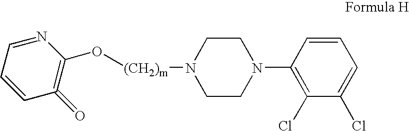
wherein m is as defined in Formula D and pharmaceutically acceptable salts thereof.

A further embodiment of the present invention is a compound of the Formula I 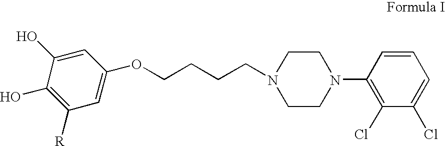
wherein R is selected from the group consisting of H, OH, CN and NO₂; and pharmaceutically acceptable salts thereof.

A further embodiment of the present invention is a compound of the Formula J

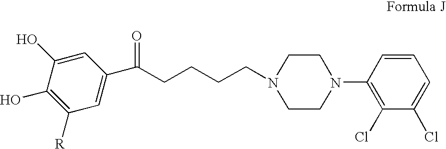

wherein R is selected from the group consisting of H, OH, CN and NO₂ and pharmaceutically acceptable salts thereof.

A further embodiment of the present invention is a compound of the Formula K 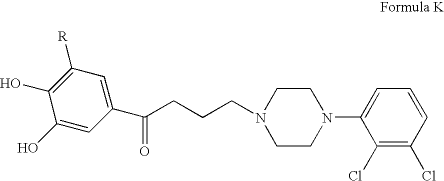
wherein R is selected from the group consisting of H, OH, CN and NO² and pharmaceutically acceptable salts thereof.

A further embodiment of the present invention is a compound of the Formula L 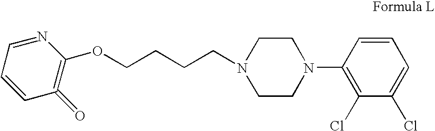
and pharmaceutically acceptable salts thereof.

A further embodiment of the present invention is a compound of the Formula M 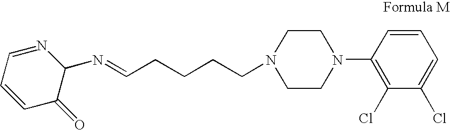
and pharmaceutically acceptable salts thereof.

A further embodiment of the present invention is a compound of the Formula N 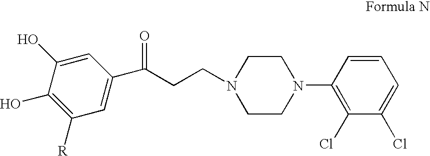
and pharmaceutically acceptable salts thereof.

A further embodiment of the present invention is a compound of the Formula O

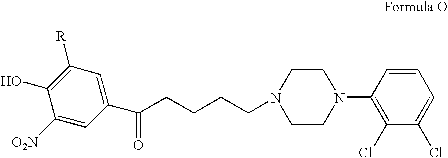

wherein R is selected from the group consisting of H, NO₂, OH, and CN and pharmaceutically acceptable salts thereof.

A further embodiment of the present invention is a compound of Formula P 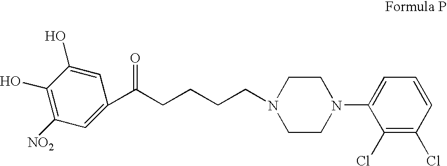
and pharmaceutically acceptable salts thereof.

A further embodiment of the present invention is a compound of the Formula Q 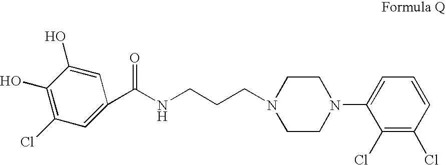
and pharmaceutically acceptable salts thereof.

A further embodiment of the present invention is a compound of the Formula R

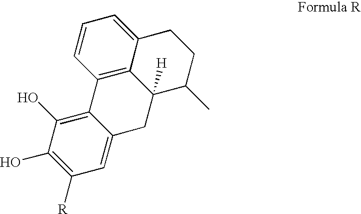

wherein R is chosen from the group consisting of H, OH, CN and NO₂ and pharmaceutically acceptable salts thereof.

A further embodiment of the present invention is a compound of the Formula S

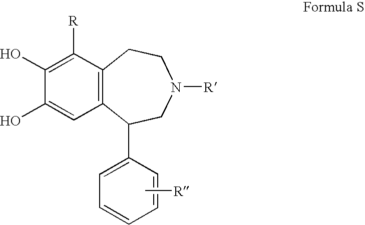

wherein R is chosen from the group consisting of OH, CN and NO₂;

R′ is chosen from the group consisting of H, C₁₋₆ alkyl and C₂₋₆ alkenyl;

R″ is chosen independently for each position capable of substitution from the group consisting of H, C₁₋₆ alkyl, halogen, hydroxyl, nitro and cyano; and enantiomers and diastereomers thereof and pharmaceutically acceptable salts thereof.

A further preferred embodiment is where Formula S, R′ is chosen from the group consisting of H, CH₃ and

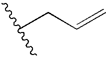

A further preferred embodiment is where in Formula S, R″ is chosen from the group consisting of H and CH₃.

Further preferred embodiment of the present invention is a compound of the Formula T

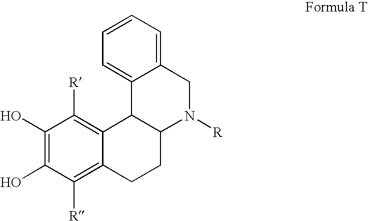

wherein R is selected from the group consisting of H and C₁₋₆ alkyl;

R′ and R″ are each independently selected from the group consisting of H, OH, CN and NO₂ with the proviso that R′=R″≠H

and enantiomers, diastereomers and pharmaceutically acceptable salts thereof.

A further preferred embodiment of the present invention is a compound of Formula U

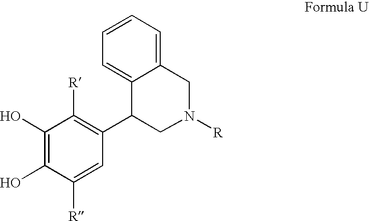

wherein R is selected from the group consisting of H and C₁₋₆ alkyl;

R′ and R″ are each independently selected from the group consisting of H, OH, NO₂ and CN with the proviso that R′=R″≠H and enantiomers and pharmaceutically acceptable salts thereof.

A further preferred embodiment of the present invention is a compound of Formula V

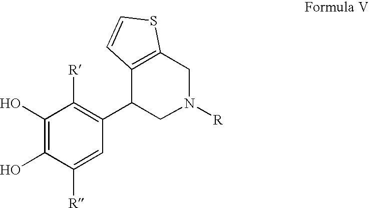

wherein R is selected from the group consisting of H and C₁₋₆ alkyl

R′ and R″ are each independently selected from the group consisting of H, OH, NO₂ and CN with the proviso that R′=R″≠H; and enantiomers and diastereomers and pharmaceutically acceptable salts thereof.

A further preferred embodiment of the present invention is a compound of Formula W

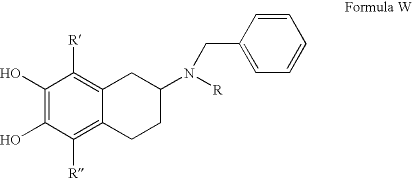

wherein R is selected from the group consisting of H and C1-6 alkyl;

R′ and R″ are each independently selected from the group consisting of H, OH, CN and NO₂ with the proviso that R′≠R″≠H and enantiomers and diastereomers thereof and pharmaceutically acceptable salts thereof.

A further preferred embodiment of the present invention is a compound of Formula X

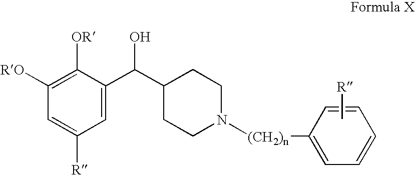

wherein R is selected from the group consisting of OH, NO₂ and CN;

R′ is selected from the group consisting of H and C₁₋₃ alkyl;

R″ is selected independently for each position capable of substitution from the group consisting of halogen, hydroxyl, hydrogen, C₁₋₅ alkyl, cyano and nitro; n is 0-6 and enantiomers and diasteroemers thereof and pharmaceutically acceptable salts thereof.

A further preferred embodiment of the present invention is a compound of Formula Y 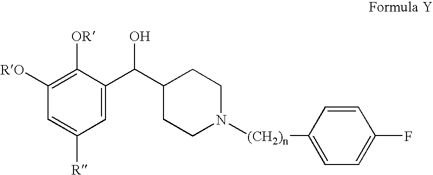
wherein R′, R″ and n are as defined in Formula X and pharmaceutically acceptable salts there.

A further embodiment is a compound according to Formula X wherein n is 2.

A further embodiment is a compound according to Formula X wherein R′ is H.

A further embodiment of the present invention is a pharmaceutical composition comprising one or more compounds according Formula A-Y in a therapeutically effective amount and in combination with one or more pharmaceutically acceptable carriers or diluents.

A further embodiment of the present invention is a method of treatment of Parkinson's Disease, Tourett's syndrome, Cognitive impairment depression, Alzheimer's disease, senile dementia, anxiety disorders, ischemic disease states, obsessive compulsive disorder, migraine, amyotrophic lateral sclerosis, epilepsy, eating disorders, premenstrual syndrome, attention deficit hyperactivity disorders, bipolar disorders, sexual dysfunction, and psychoses comprising administering to a patient in need of said treatment a pharmaceutical composition comprising a compound accordin to Formulas A-Y.

A further embodiment of the present invention is a method for the treatment of schizophrenia comprising administering to a patient in need of said treatment a pharmaceutical composition comprising a compound according to Formulas A-Y.

A further embodiment of the present invention is a method for the treatment of the positive symptoms of schizophrenia comprising administering to a patient in need of such treatment a pharmaceutical composition comprising a compound according to Formulas A-Y.

A further embodiment of the present invention is a method for the treatment of the negative symptoms of schizophrenia comprising administering to a patient in need of such treatment a pharmaceutical composition comprising a compound according to Formulas A-Y.

Examples 1-11 provide the starting materials and generalized reaction pathways for the synthesis of compounds having pharmacophores active at inhibiting COMT and simultaneously acting at dopamine receptors as described in the preceding paragraph. Compounds that fulfill this dual or triple functionality are illustrated as structures 1-108. Analogs of these structures apparent to one skilled in the art are also claimed.

Several compounds were synthesized using the principals outlined in examples 1-11. The structure of the rationally designed compounds and their synthetic pathways are provided in examples 12 and 13.

The present invention further provides a paradigm for the prioritization of compounds having the ability to ameliorate the positive and negative symptoms of schizophrenia, and improve mild cognitive impairments. The paradigm may be used to evaluate rationally described compounds as synthesized e.g. in examples 12 and 13. This screening method can also be used to evaluate compounds not here-to-fore identified as useful for the treatment of schizophrenia. The method is based on the observation that simultaneous D2 receptor partial agonism in the striatum and COMT inhibition in the frontal cortex are likely to provide a beneficial effect in patients suffering from the positive and negative symptoms of schizophrenia. In addition to these activities, compounds particularly suitable for use in the treatment of schizophrenia may also demonstrate D1 agonism Example 14 provides methods for the execution of this strategy. A diagrammatic description of the screening method is given in FIG. 3

It is understood that the prioritization method described in this invention may also be used to identify compounds acting at only one or several, but not all, of the specificities described above. It is also envisioned that synthetic pathways may not be devisable which can combine certain compounds that, when co-administered to a patient, have the collective effect of treating both the positive and negative side effects of schizophrenia. The scope of this invention further encompasses the administration of one or several compounds that have the combined effect of treating the positive and negative side effects of schizophrenia

The compounds synthesized as described in examples 12-13 were evaluated using several of the principals described in example 14. Competitive binding assays utilizing receptors critical for the proper functioning of the drugs were performed. Experiments involving receptor binding to the dopamine D1, dopamine D2, 5-HT1A and 5-HT2A receptors as described in example 14 are graphically represented in FIGS. 5 and 6. In addition, the ability of compounds PGX-2000001 to PGX 2000004 to inhibit COMT was evaluated. Example 12 illustrates the experimental protocol for determining COMT inhibition.

### BRIEF DESCRIPTION OF THE FIGURES

FIG. 1. Proposed actions and clinical benefits of a D2 partial agonist-COMT inhibitor on the release and metabolism of dopamine (symbolized by small circles) and its availability at pre- and post-synaptic receptors (symbolized by hexagons) in the frontal cortex. Dopamine autoreceptors and reuptake are believed to play a relatively small role in regulating dopamine in the frontal cortex and thus have been omitted from the illustration. Similar effects of COMT on augmenting norepinephrine signaling in the frontal cortex is also expected as norepinephrine is also a substrate for COMT.

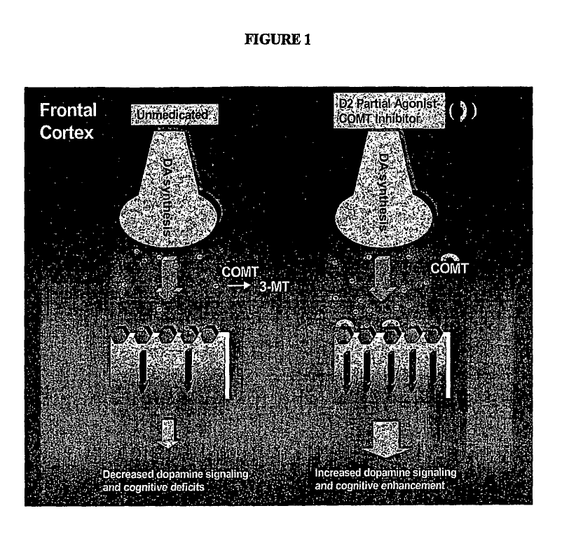

FIG. 2. Proposed actions and clinical benefits of a D2 partial agonist-COMT inhibitor on the release and metabolism of dopamine (symbolized by small circles) and its availability at pre- and post-synaptic receptors (symbolized by hexagons) in the striatum, where dopamine reuptake exceeds COMT as the main route of inactivating dopamine.

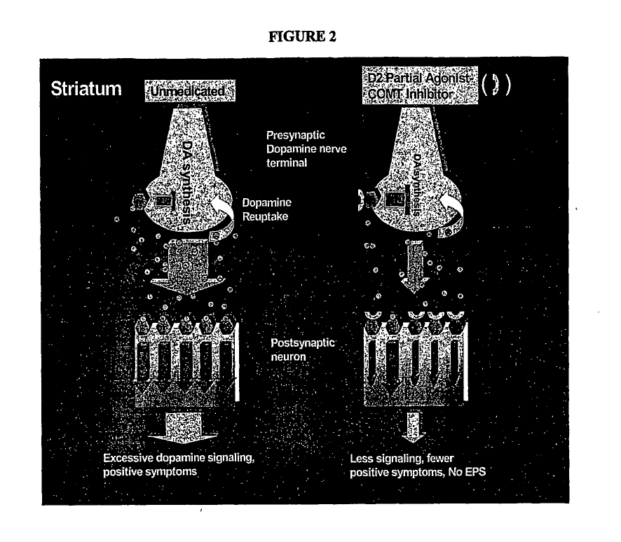

FIG. 3. Schematic illustration of methods used for identifying and prioritizing lead compounds effective at treating the positive and negative side effects of schizophrenia

FIG. 4. Results of receptor binding assays. Compounds designated as PGX 2000001 and PGX 2000002 were evaluated for their ability to displace competitively binding compounds at the D1, D2, 5HT1A and 5HT2A receptors

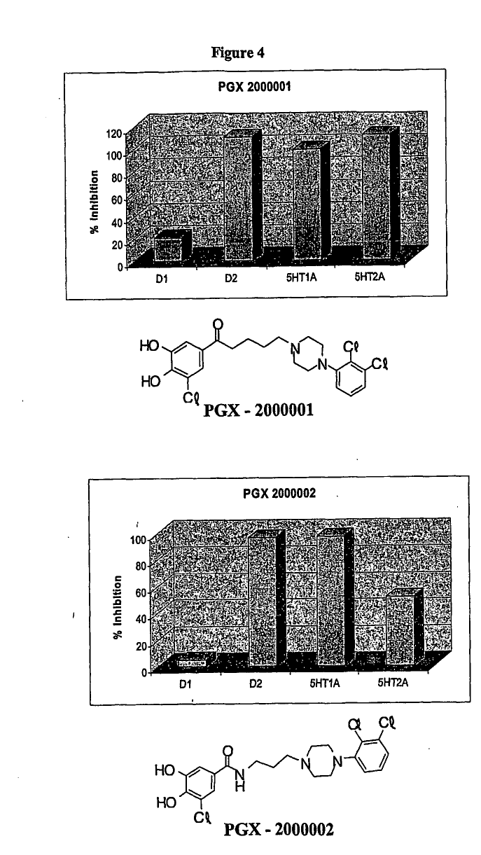

FIG. 5. Results of receptor binding assays. Compounds designated as PGX 2000003 and PGX 2000004 were evaluated for their ability to displace competitively binding compounds at the D1, D2, 5HT1A and 5HT2A receptors

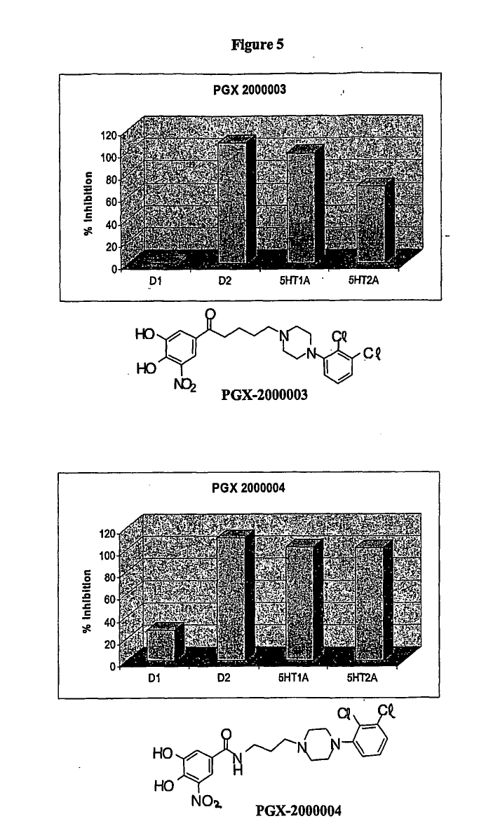

FIG. 6. Results of receptor binding assays. The compound aripiprazole and tolcapone were evaluated for their ability to displace competitively binding compounds at the D1, D2, 5HT1A and 5HT2A receptors.

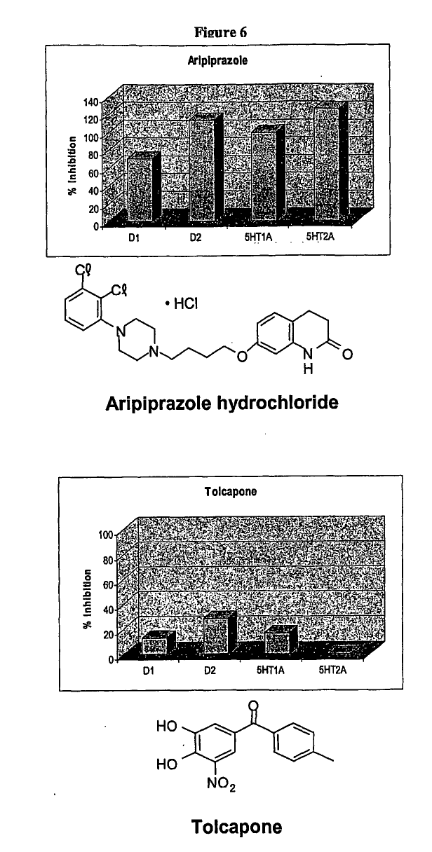

FIG. 7. Inhibition of COMT by aripiprazole, tolcapone and PGX compounds 1-4. The compounds effects were tested at concentration of 10 and 50 μM.

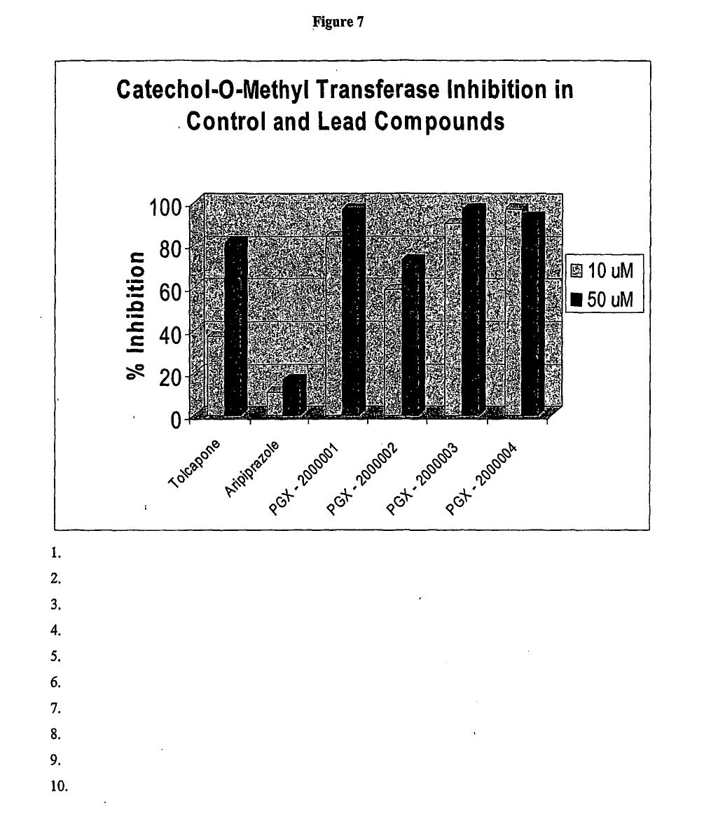

### DETAILED DESCRIPTION OF INVENTION

The term ‘alkyl’ refers to a straight or branched hydrocarbon chain radical consisting solely of carbon and hydrogen atoms, containing solely of carbon and hydrogen atoms, containing no unsaturation, having from one to eight carbon atoms, and which is attached to the rest of the molecule by a single bond, e.g., methyl, ethyl, n-propyl, 1-methylethyl (isopropyl), n-butyl, n-pentyl, 1,1-dimethylethyl (t-butyl), and the like.

The term “Alkenyl” refers to aliphatic hydrocarbon group containing a carbon-carbon double bond and which may be a straight or branched or branched chain having about 2 to about 10 carbon atoms in the e.g., ethenyl, 1-propenyl, 2-propenyl (allyl), iso-propenyl, 2-methyl-1-propenyl, 1-butenyl, 2-butenyl and the like.

The term “Alkoxy” denotes alkyl group as defined above attached via oxygen linkage to the rest of the molecule. Representative examples of those groups are —OCH₃, —OC₂H₅ and the like.

The term “Halogen” refers to radicals of Fluorine, Chlorine, Bromine, Iodine

“Treating” or “treatment” of a state, disorder or condition includes:
(1) preventing or delaying the appearance of clinical symptoms of the state, disorder or condition developing in a mammal that may be afflicted with or predisposed to the state, disorder or condition but does not yet experience or display clinical or subclinical symptoms of the state, disorder or condition;
(2) inhibiting the state, disorder or condition, i.e., arresting or reducing the development of the disease or at least one clinical or subclinical symptom thereof; or
(3) relieving the disease, i.e., causing regression of the state, disorder or condition or at least one of its clinical or subclinical symptoms.

The benefit to a subject to be treated is either statistically significant or at least perceptible to the patient or to the physician.

A “therapeutically effective amount” means the amount of a compound that, when administered to a mammal for treating a state, disorder or condition, is sufficient to effect such treatment. The “therapeutically effective amount” will vary depending on the compound, the disease and its severity and the age, weight, physical condition and responsiveness of the mammal to be treated.

“Delivering” a therapeutically effective amount of an active ingredient to a particular location within a host means causing a therapeutically effective blood concentration of the active ingredient at the particular location. This can be accomplished, e.g., by local or by systemic administration of the active ingredient to the host.

“A subject” or “a patient” or “a host” refers to mammalian animals, preferably humans.

Pharmaceutically acceptable salts forming part of this invention include salts derived from inorganic bases such as Li, Na, K, Ca, Mg, Fe, Cu, Zn, Mn; salts of organic bases such as N,N′-diacetylethylenediamine, glucamine, triethylamine, choline, choline hydroxide, dicyclohexylamine, metfornin, benzylamine, trialkylaamine, thiamine, spermidine, and the like; chiral bases like alkylphenylamine, glycinol, phenyl glycinol and the like, salts of natural amino acids such as glycine, alanine, valine, leucine, isoleucine, norleucine, tyrosine, cystine, cysteine, methionine, proline, hydroxy proline, histidine, omithine, lysine, arginine, serine, and the like; unnatural amino acids such as D-isomers or substituted amino acids; guanidine, substituted guanidine wherein the substituents are selected from nitro, amino, alkyl, alkenyl, alkynyl, ammonium or substituted ammonium salts and aluminum salts. Salts may include acid addition salts where appropriate which are, sulphates, nitrates, phosphates, perchlorates, borates, hydrohalides, acetates, tartrates, maleates, citrates, succinates, palmoates, methanesulphonates, benzoates, salicylates, benzenesulfonates, ascorbates, glycerophosphates, ketoglutarates and the like. Pharmaceutically acceptable solvates may be hydrates or comprise other solvents of crystallization such as alcohols.

Various polymorphs of a compound of forming part of this invention may be prepared by crystallization of compound forming part of this invention under different conditions, example, using different solvents commonly used or their mixtures for recrystallization; crystallizations at different temperatures, various modes of cooling, ranging from very fast to very slow cooling during crystallizations. Polymorphs may also be obtained by heating or melting the compound followed by gradual or fast cooling. The presence of polymorphs may be determined by solid probe nmr spectroscopy, IR spectroscopy, differential scanning calorimetry, powder X-ray diffiraction or such other techniques.

The present invention provides compounds, their analogs, their tautomers, their regioisomers, their stereoisomers, their enantiomers, their diastreomers, their polymorphs, their pharmaceutically acceptable salts, and their pharmaceutically acceptable solvates.

The present invention also provides pharmaceutical compositions, containing compounds of the invention, their derivatives, their analogs, their tautomeric forms, their stereoisomers, their polymorphs, their enantiomers, their diasteromers, their pharmaceutically acceptable salts or their pharmaceutically acceptable solvates in combination with the usual pharmaceutically employed carriers, diluents and the like. The pharmaceutical compositions according to this invention can be used for the treatment of A method of treatment of Parkinson's Disease, Tourett's syndrome, Cognitive impairment, depression, Alzheimer's disease, senile dementia, anxiety disorders, ischemic disease states, obsessive compulsive disorder, migraine, amyotrophic lateral sclerosis, epilepsy, eating disorders, premenstrual syndrome, attention deficit hyperactivity disorders, bipolar disorders, sexual dysfunction, schizophrenia and positive and negative effects of schizophrenia and psychoses.

It will be appreciated that some of the compounds according to the invention can contain one or more asymmetrically substituted carbon atoms. The presence of one or more of these asymmetric centers in the compounds of the invention can give rise to stereoisomers and in each case the invention is to be understood to extend to all such stereoisomers, including enantiomers and diastereomers and their mixtures, including racemic mixtures.

The pharmaceutical compositions may be in the forms normally employed, such as tablets, capsules, powders, syrups, solutions, suspensions and the like and may contain flavorants, sweeteners etc. in suitable solid or liquid carriers or diluents, or in suitable sterile media to form injectable solutions or suspensions. The active compounds of the invention will be present in such pharmaceutical compositions in the amounts sufficient to provide the desired dosage in the range as described above. Thus, for oral administration, the compounds of the invention can be combined with a suitable solid, liquid carrier or diluent to form capsules, tablets, powders, syrups, solutions, suspensions and the like. The pharmaceutical compositions, may, if desired, contain additional components such as flavorants, sweeteners, excipients and the like. For parenteral administration, the compounds of the invention can be combined with sterile aqueous or organic media to form injectable solutions or suspensions. For example, solutions in sesame or peanut oil, aqueous propylene glycol and the like can be used as well as aqueous solutions of water-soluble pharmaceutically-acceptable acid addition salts or salts with base of the compounds of the invention. The injectable solutions prepared in this manner can then be administered intravenously, intraperitoneally, subcutaneously, or intramuscularly, with intramuscular administration being preferred in humans.

In addition to the compounds of the invention pharmaceutical compositions of the present invention may also contain or be co-administered with one or more known drugs selected from other clinically useful therapeutic agents.

The invention is described here, in detail, by way of particular examples. However, the use of such examples is illustrative only and in no way limits the scope or meaning of this invention or of any exemplified term. Nor is the invention limited to any particular embodiments described in these examples. Indeed, many modifications and variations of the invention will be apparent to those skilled in the art upon reading this specification, and such equivalents can be made without departing from the invention in spirit or in scope. The invention is therefore limited only by the terms of the appended claims along with the full scope of equivalents to which the claims are entitled.

A basis for the instant invention is the observation that dopamine signaling in distinct regions of the brain is modulated based on different mechanisms of control. Turnover and concentration of dopamine in the synapses of striatal neurons is more heavily influenced by the relatively high abundance of dopamine receptors and dopamine transporters. In the cortex, COMT plays the most prominent role in.

Yet another basis of the invention resides in the observation that multiple receptors and enzymes are druggable targets useful for the treatment of schizophrenia. These targets include receptors such as the 5-HT1A and 5-HT2A receptors, the dopaminergic receptors and enzymes such as catechol-o-methyl transferase. Drug action at a combination of these receptors and COMT targets is likely to modulate the positive or negative side effects of schizophrenia (including cognitive functioning). The most successful drugs will affect more than one site relevant to the biology of schizophrenia.

### EXAMPLE 1

The starting point for synthesis of the novel dual action COMT inhibitor/D2 partial agonist compounds is a portion of a D2 receptor-interacting drug such as Aripiprazole (see 1 below).

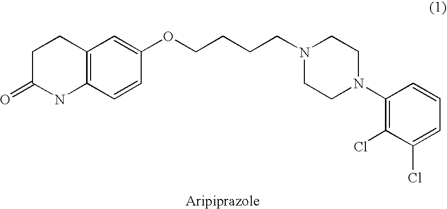

In the case of the dual action molecules, the left hand side of the molecule is replaced with the active components of molecules in the entacapone (2) series. Compounds in the series include the centrally acting catechol-o-methyltransferase inhibitor, tolcapone.

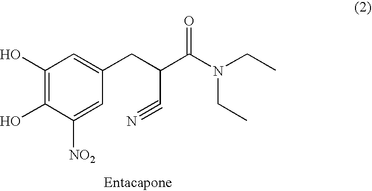

These molecules have structures such as (3) below:

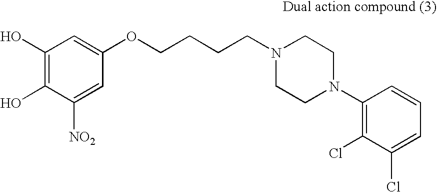

This can be generalized to the compound series (4) below:

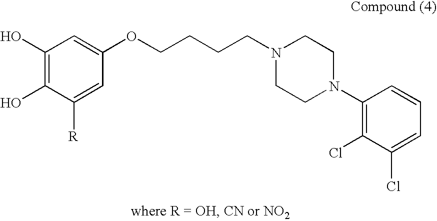

This includes propyl gallate-like structures (R=OH) or the cyano series of compounds (R=CN) such as OR-490 and OR-657. Compounds with R=NO₂ are similar to Entacapone, Nitecapone and Tolcapone.

Further expansion of the series includes structures with a keto functionality proximal to the LHS phenyl ring. Examples are (5) or (6):

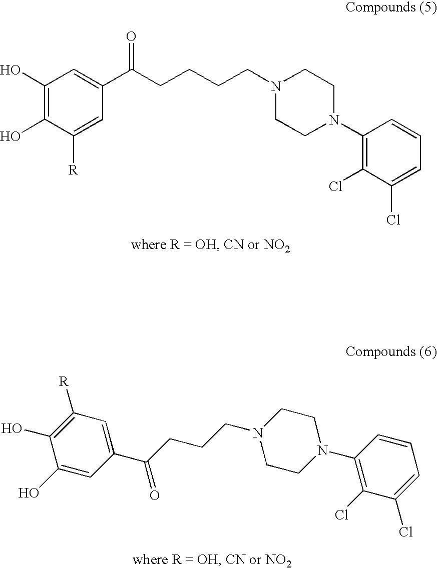

### EXAMPLE 2

Further elaboration of the LHS of the molecule includes the substructure from CGP 28014 (7) below:

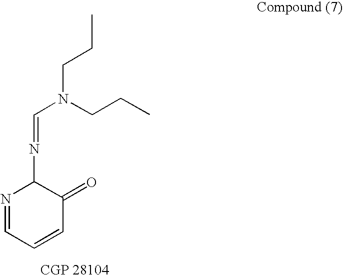

This leads to structures such as compounds (8) or (9) below:

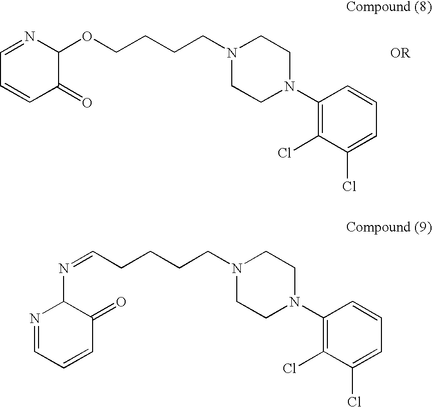

### EXAMPLE 3

A completely different set of dual active compounds call also be created from fusion of the aripiprazole structure with that suggested by Bonifacio (10) below:

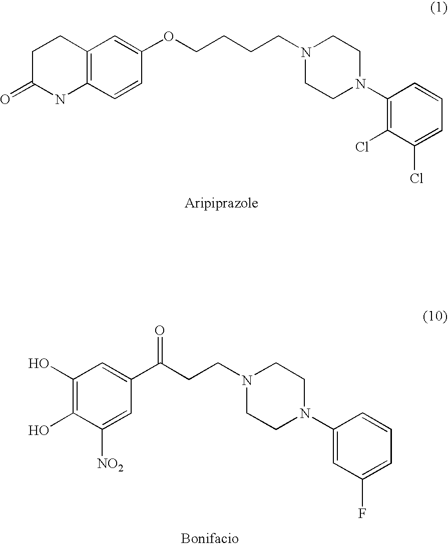

Dual active compounds 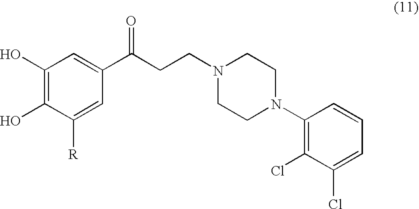
(Where R can include NO₂, OH, or CN) or with 1 further carbon in the chain to more closely mimic Aripiprazole (see below). 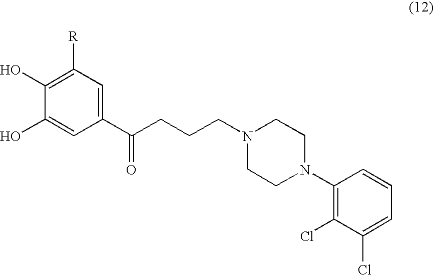
(Where R=NO₂, OH, or CN)

### EXAMPLE 4

A set of compounds with dual dopamine D1 agonist/D2 partial agonist activity based on apomorphine (13) can also be synthesized. Addition of COMT inhibition can be achieved as shown in the following molecules by addition of the R group (R=OH, CN or NO₂) (compounds 14).

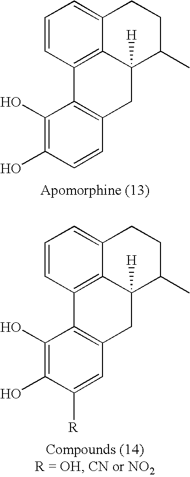

R=OH, CN or NO₂

### EXAMPLE 5

A set of compounds based on the agonist and partial agonist dopamine D1 properties of SKF 38393(15), SKF 82958(16) and SKF 83959(17). These compounds are in the classes (18), (19) and (20), respectively.

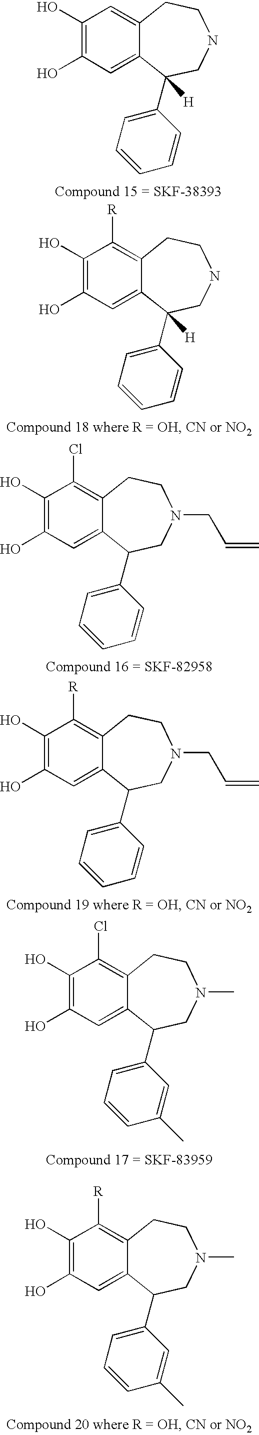

### EXAMPLE 6

A set of compounds following similar COMT inhibiting principles can also be based on the D1 agonist properties of A-86929, ABT-431 (adroglide HCl) and dihexedrine.

A series of compounds can be realized as dual acting compounds derived from dihydrexidine (21-23) (Brewster, W. K. et al., J Med Chem (1990) 33, 1756-64)

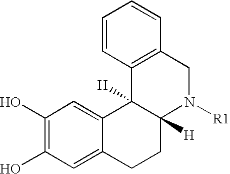

Compounds 21-23 where R1=H, CH₃, n-C₃H₇
Derived compounds have the structure 24-32.

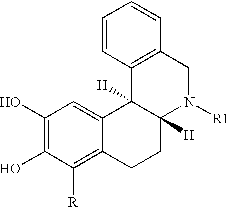

Compounds 24-32 (R=OH, NO₂, CN) and (R1=H, CH₃, n-C₃H₇)

Although the McDermed model (Mcdermed, J. D. et al., Catecholamines (1978) 568-570) suggests that compounds where R is not=H have no activity, dual acting compounds with only reduced dopamine D1 agonist activity still can fill the desired type of dual COMT inhibitor/dopamine agonist-partial agonist activity. The parent structures 21-23 all have sub uM potency at D1 and D2 receptors.

Alternate structures that have the dual activity and still meet the McDermed model include compounds such as (33-41):

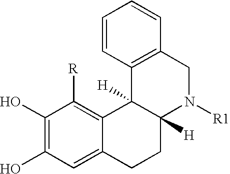

Compounds 33-41 (R=OH, NO₂, CN) and (R1=H, CH₃, n-C₃H₇)

### EXAMPLE 7

Conformationally mobile analogs active as agonists/partial agonists at the D1/D2 receptor from the series of 4-phenyl-1,2,3,4-tetrahydroisoquinolines e.g. 42 below can be considered a starting point for dual COMT/D1-D2 agonists.

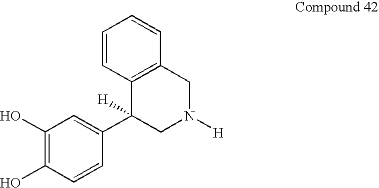

Such dual compounds have structures such as 43-51 or 52-60 below:

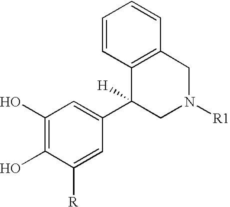

Compounds 43-51 (R=OH, NO₂, CN) and R1=H, CH₃, n-C₃H₇)

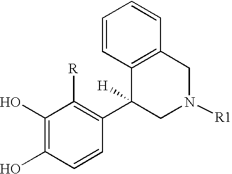

Compounds 52-60 (R=OH, NO₂, CN) and (R1=H, CH₃, n-C₃H₇)

It is likely that the McDermed rules will not apply to these compounds because these compounds are not conformationally rigid.

### EXAMPLE 8

An additional set of compounds that show dopamine D1/D2 activity are the thiophene bioisosteres of compounds related to compound 42 above. Such a thiophene bioisostere is illustrated by compound 61 (compounds 62-70 or compounds 71-79 below):

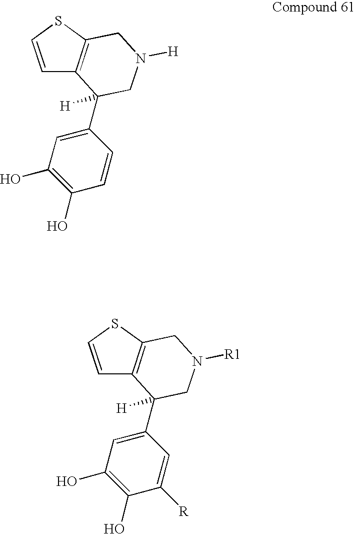

Compounds 62-70 (R=OH, NO₂, CN) and (R1=H, CH₃, n-C₃H₇)

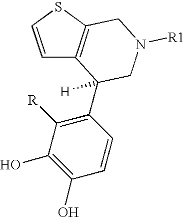

Compounds 71-79 (R=OH, NO₂, CN) and (R1=H, CH₃, n-C₃H₇)

### EXAMPLE 9

Also for consideration are compounds derived from the following compound, (also see (Brewster, W. K. Nichols, D. E., J Med Chem (1990) 33, 1756-64)) compound 80:

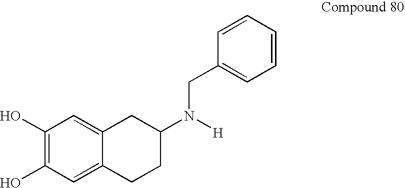

Although compound 80 is not potent enough at the D1 receptor to be considered useful by itself as an agonist, the residual partial D2 agonist activity in combination with COMT inhibitor activity can still be useful for treatment of schizophrenia.

Such compounds would have the structure of compounds 81-89 or compounds 90-98 below:

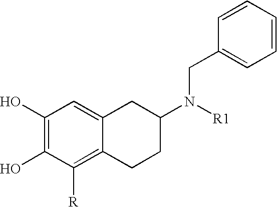

Compounds 81-89 (R=OH, NO₂, CN) and (R1=H, CH₃, n-C₃H₇)

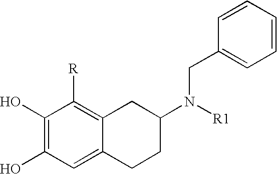

Compounds 90-98 (R=OH, NO₂, CN) and (R1=H, CH₃, n-C₃H₇)

### EXAMPLE 10

An approach to the generation of dual activity compounds involves the hypothesis that supports 5-HT-2A receptor antagonism as useful to treat the negative symptoms of psychosis (Palfreyman, M. G. et al., Psychopharmacology (Berl) (1993) 112, S60-7, De Paulis, T., Curr Opin Investig Drugs (2001) 2, 123-32). This is illustrated by MDL-100907 (compound 99).

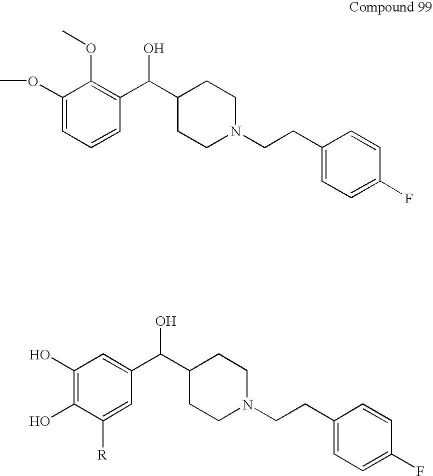

Compounds 100-108 R=OH, NO₂, CN) and (R1=H, CH₃, n-C₃H₇)

Synthetic Routes for Compounds 3-108 and Additional Novel Compounds

Synthesis of Compound (3): 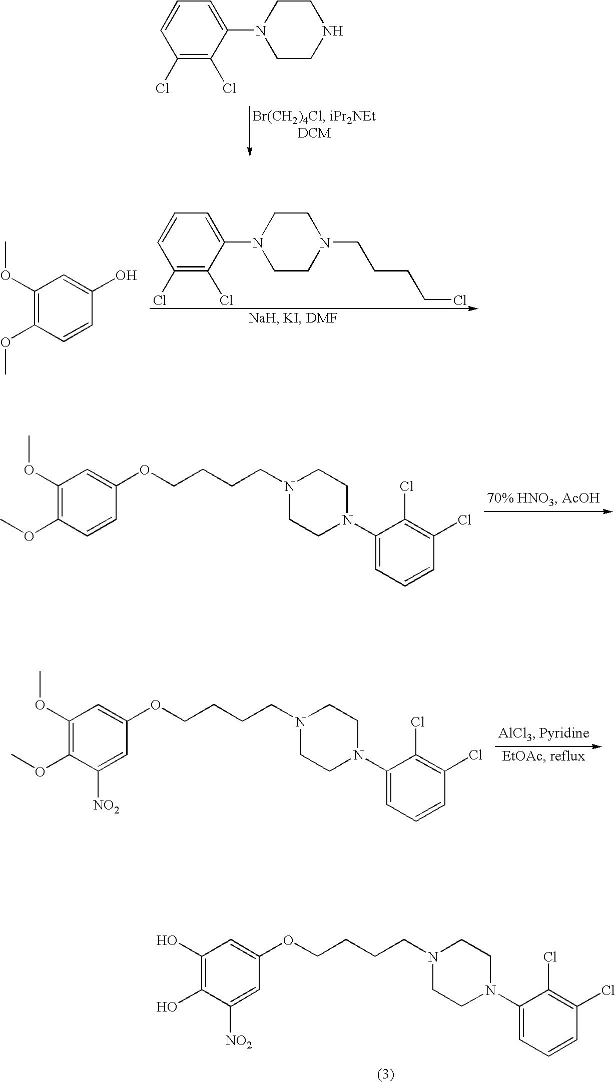
Additionally, compounds with various lengths of linker can be synthesized as shown: 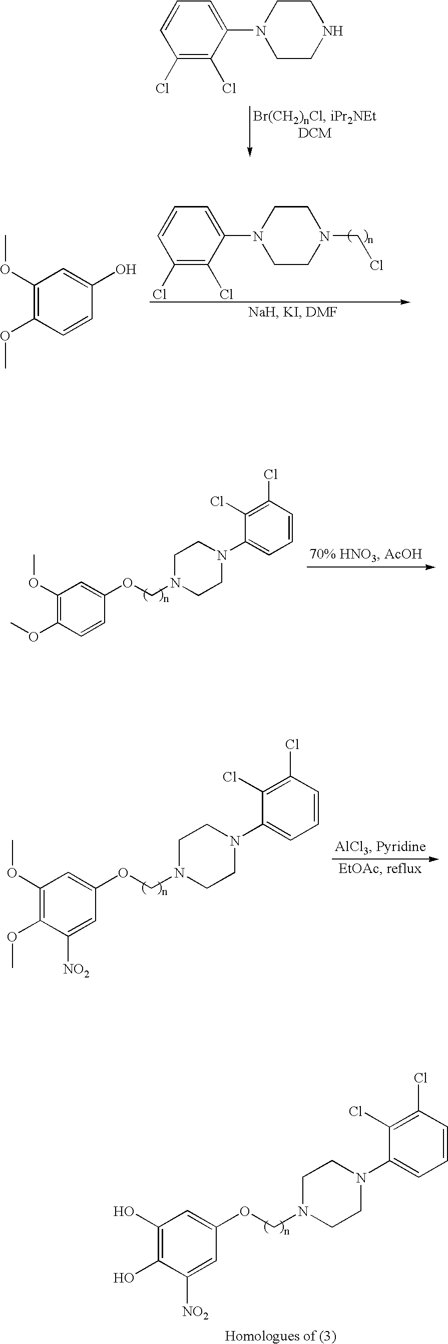
Synthesis of Compound (4), R=OH: 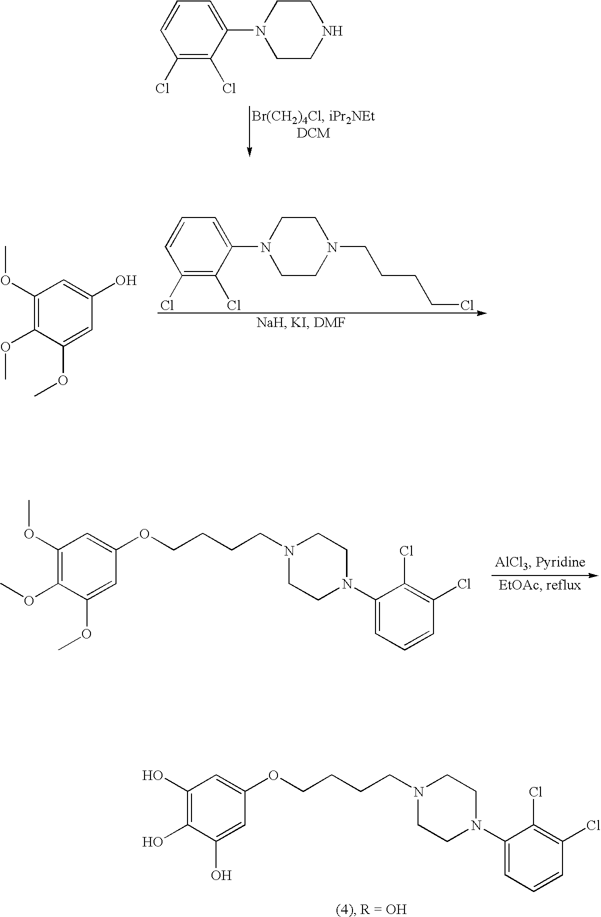
Synthesis of Homologues of Compound (4) with R=OH: 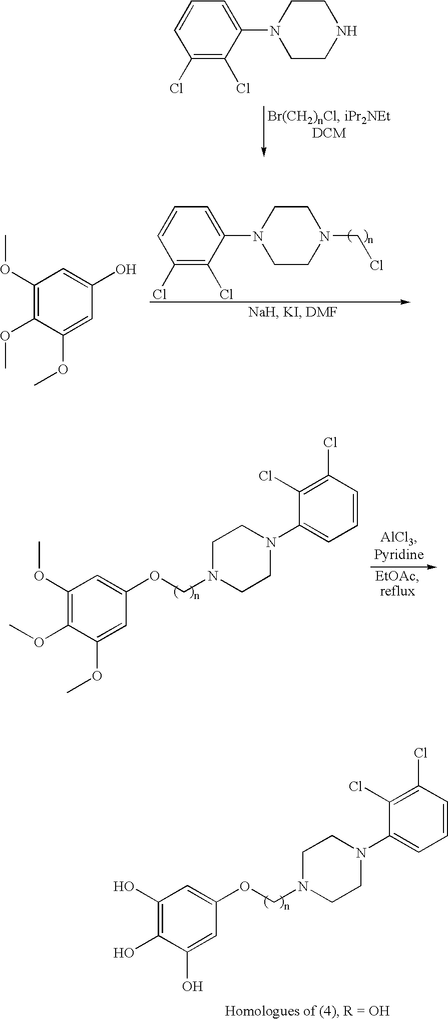
Synthesis of Compound (4), R=CN: 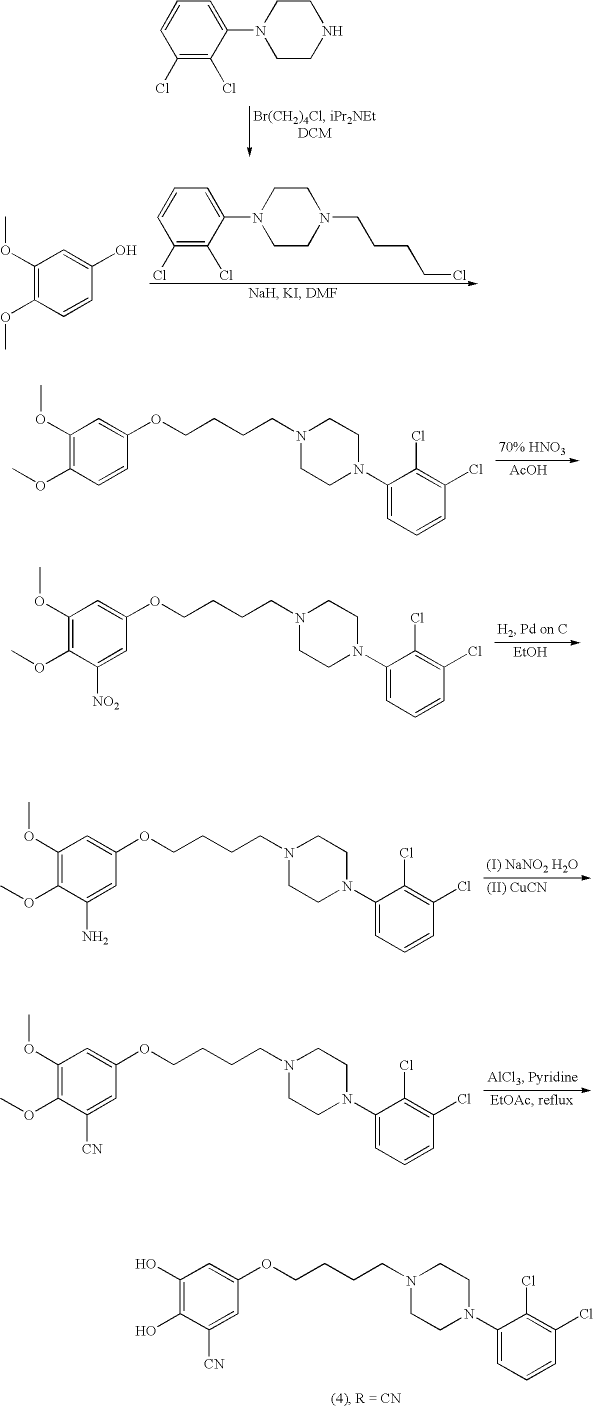
Synthesis of Homologues of Compound (4), R=CN: 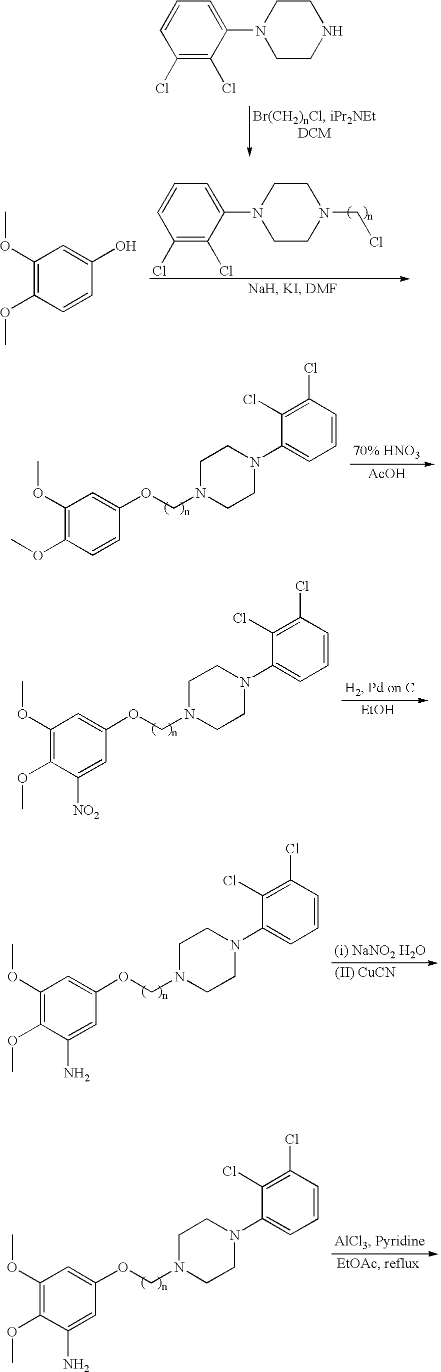 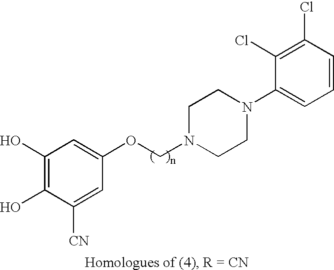
Synthesis of Compound (5), R=NO₂: 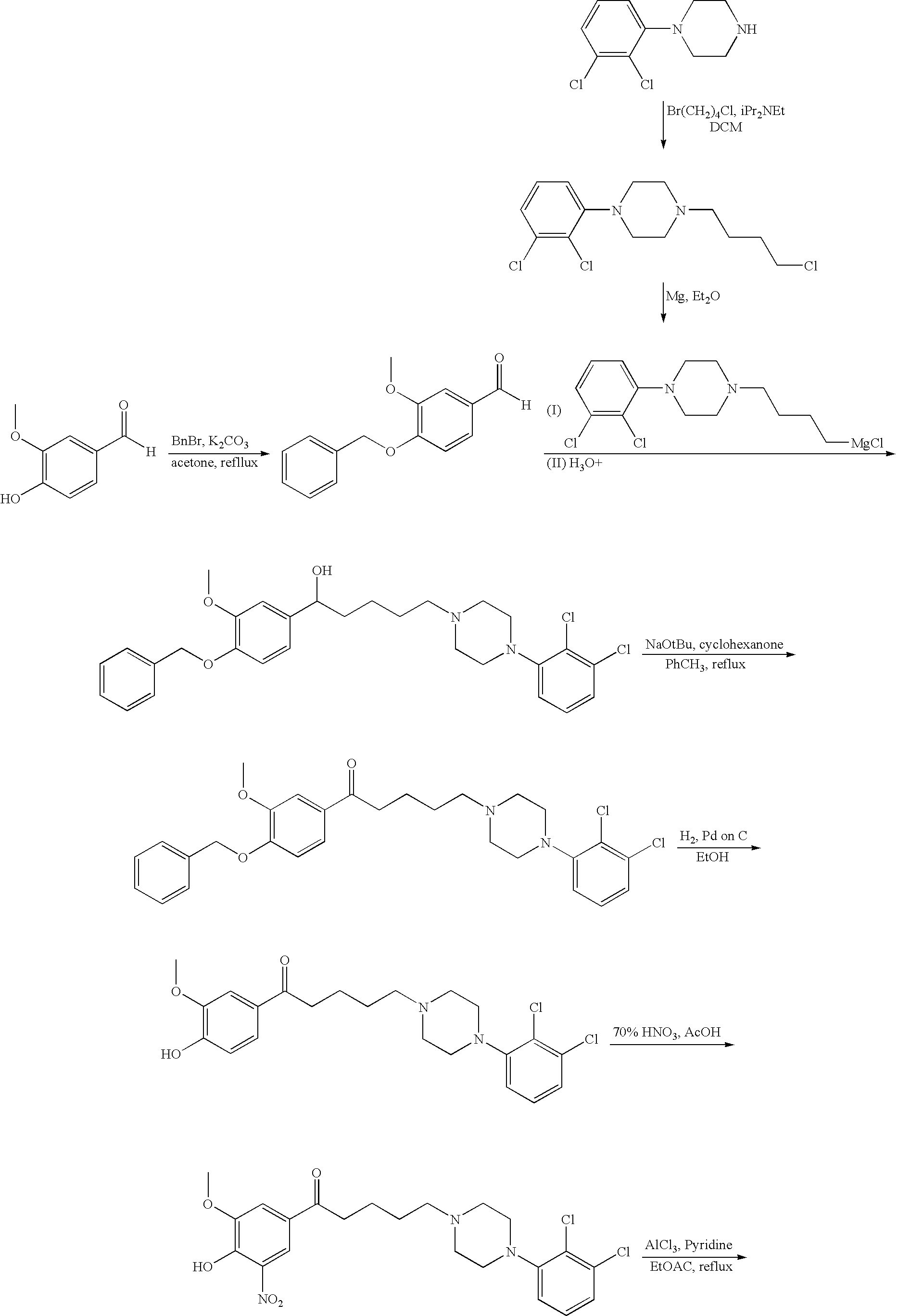 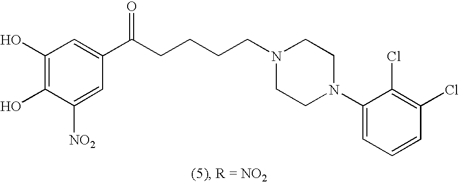
Synthesis of Homologues of Compound (5), R=NO₂: 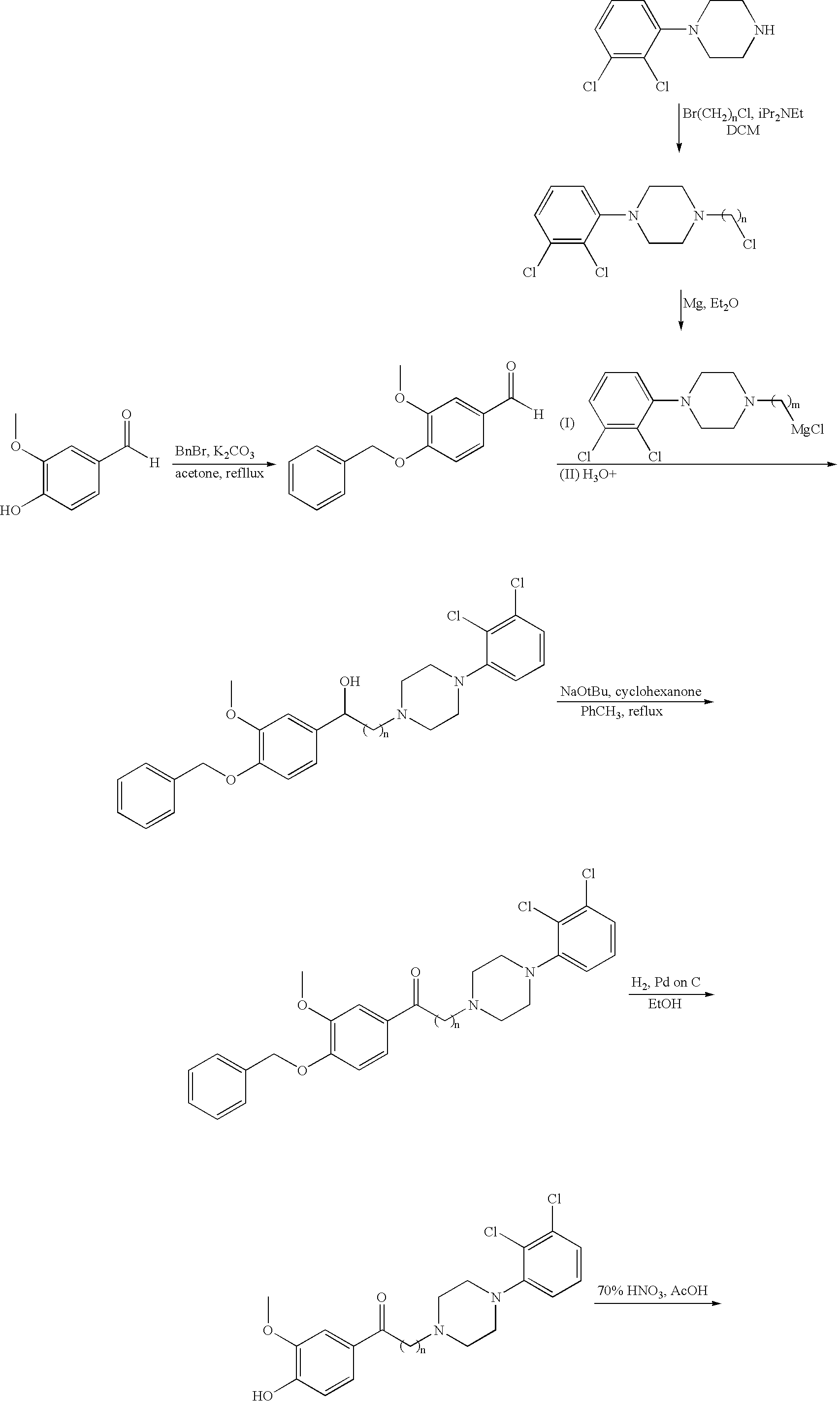 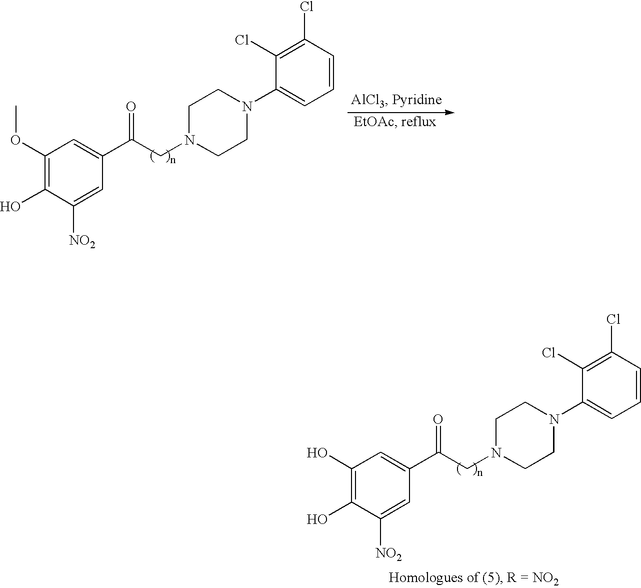
Synthesis of Compound (5), R=OH: 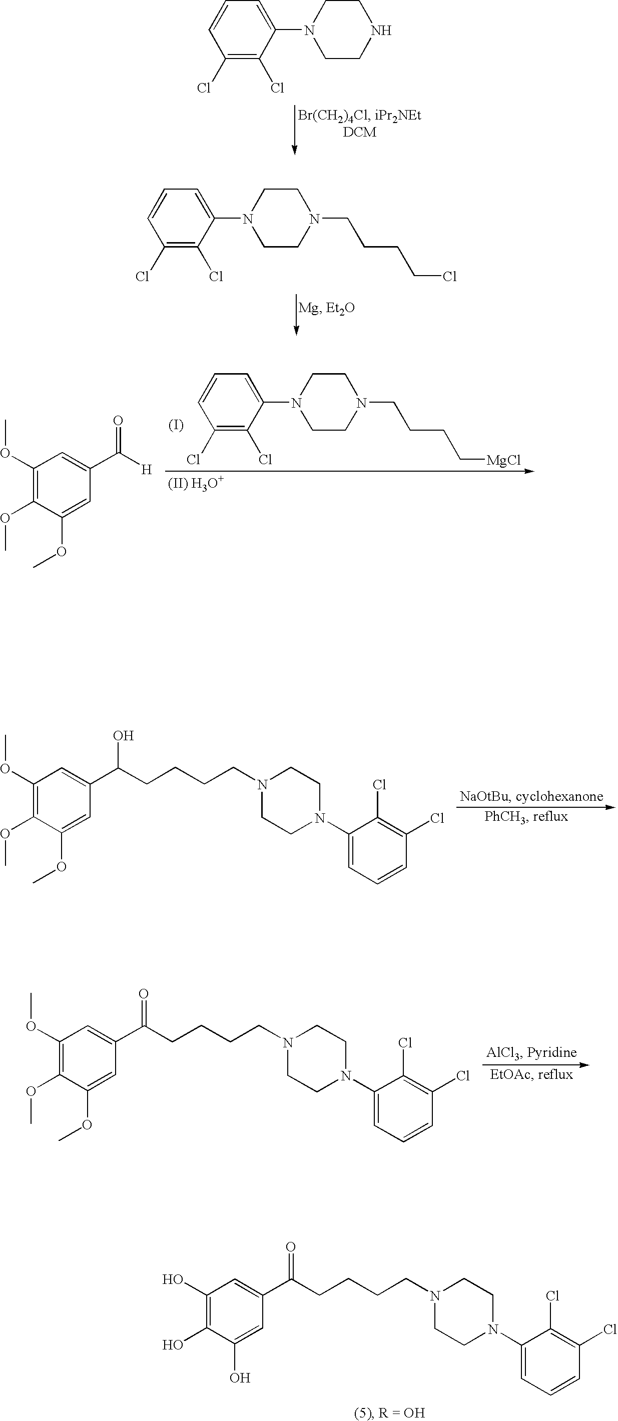
Synthesis of Homologues of Compound (5), R=OH: 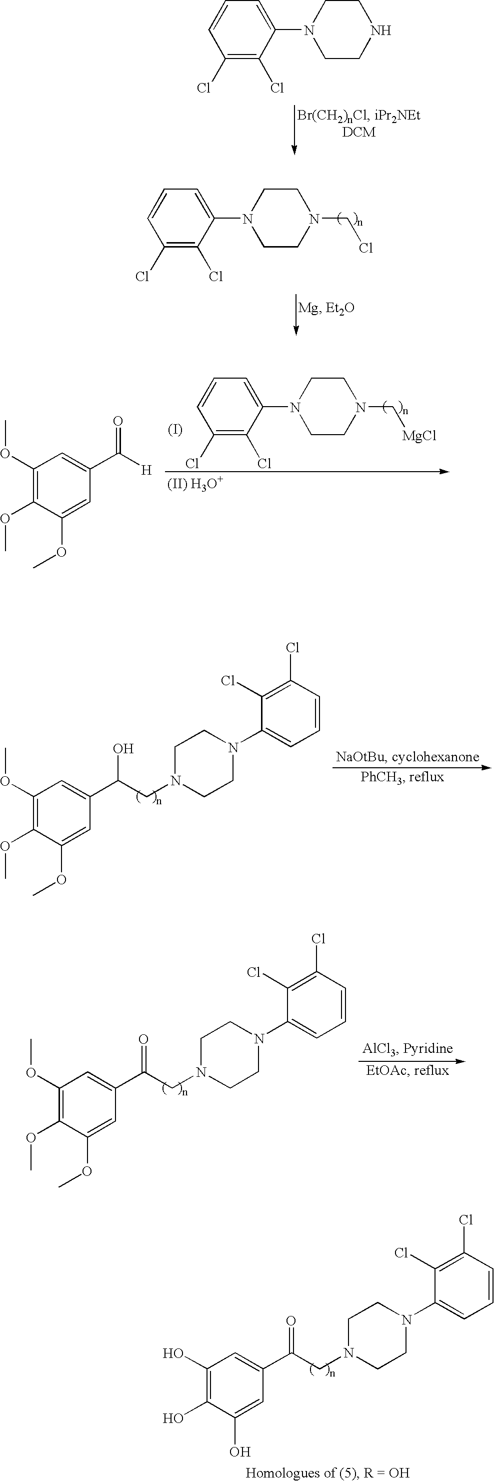
Synthesis of Compound (5), R=CN: 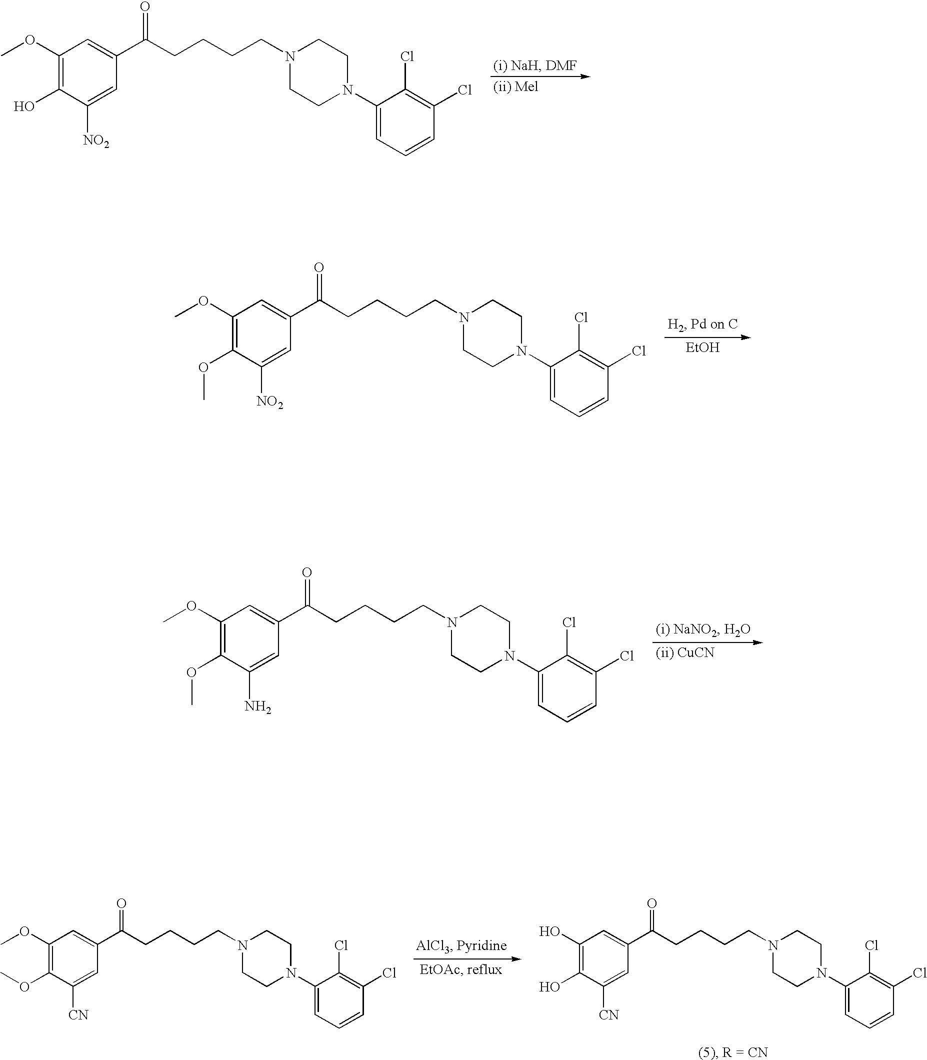
Synthesis of Homologues of Compound (5), R=CN: 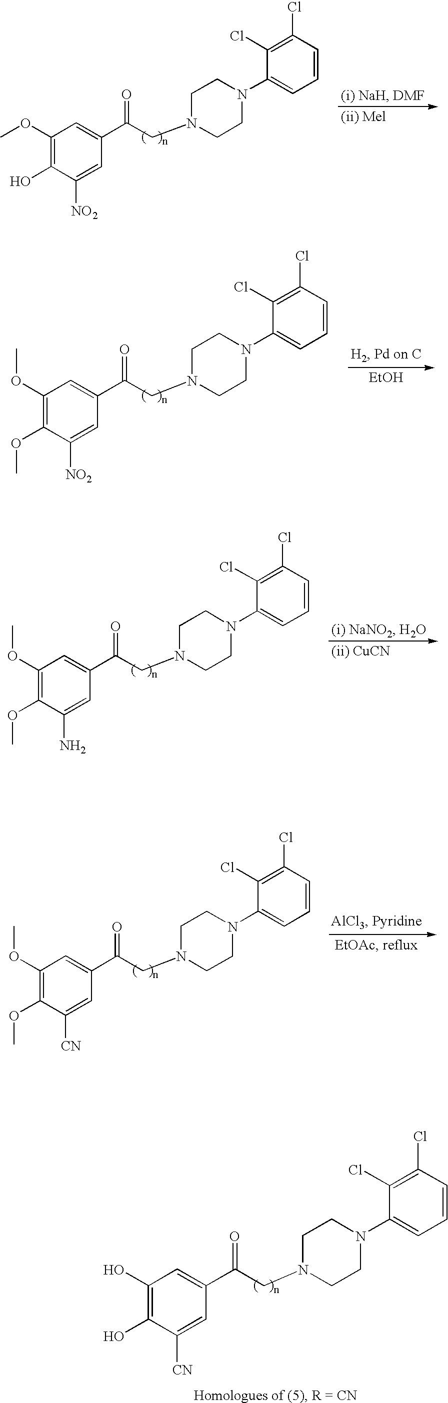
Synthesis of Compound (6), R=NO₂: 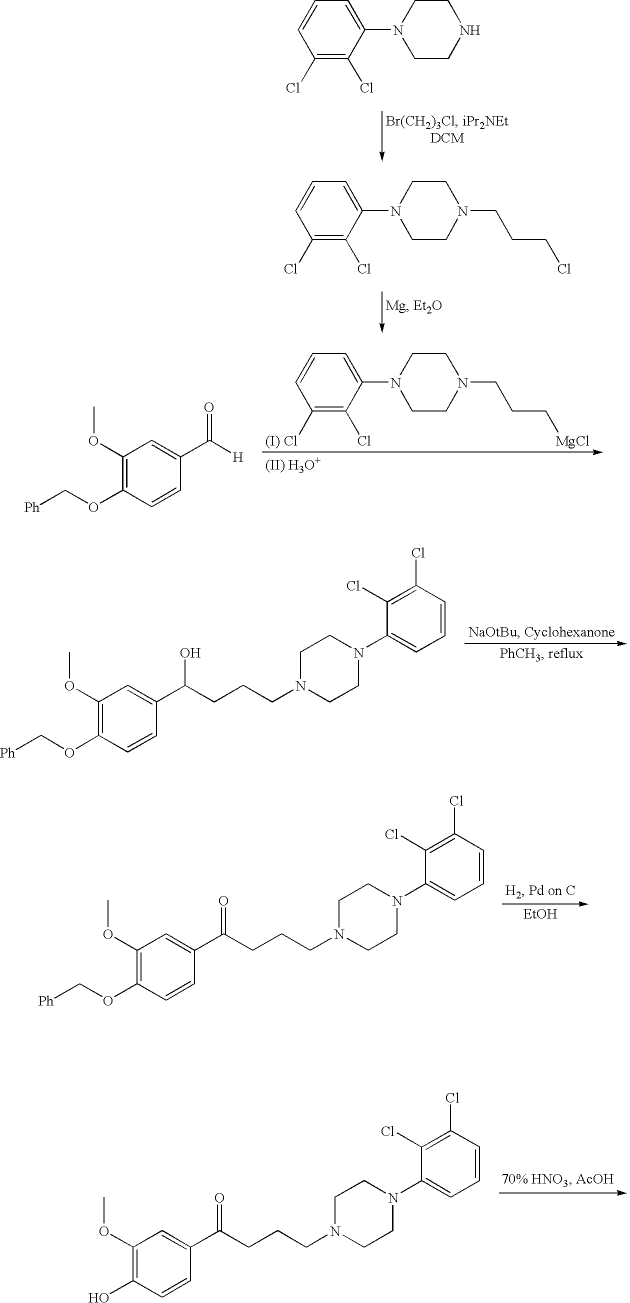 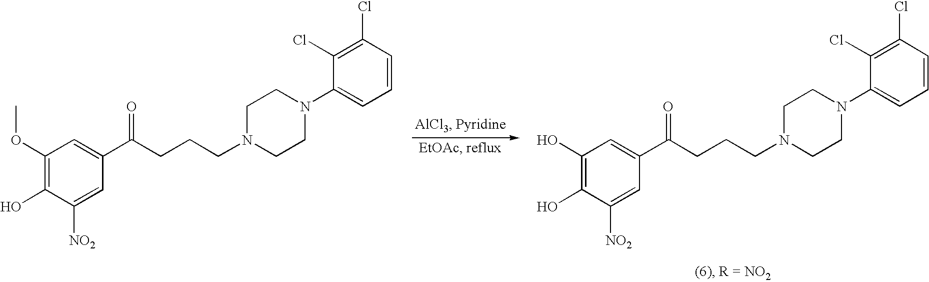
Synthesis of Compound (6), R=OH: 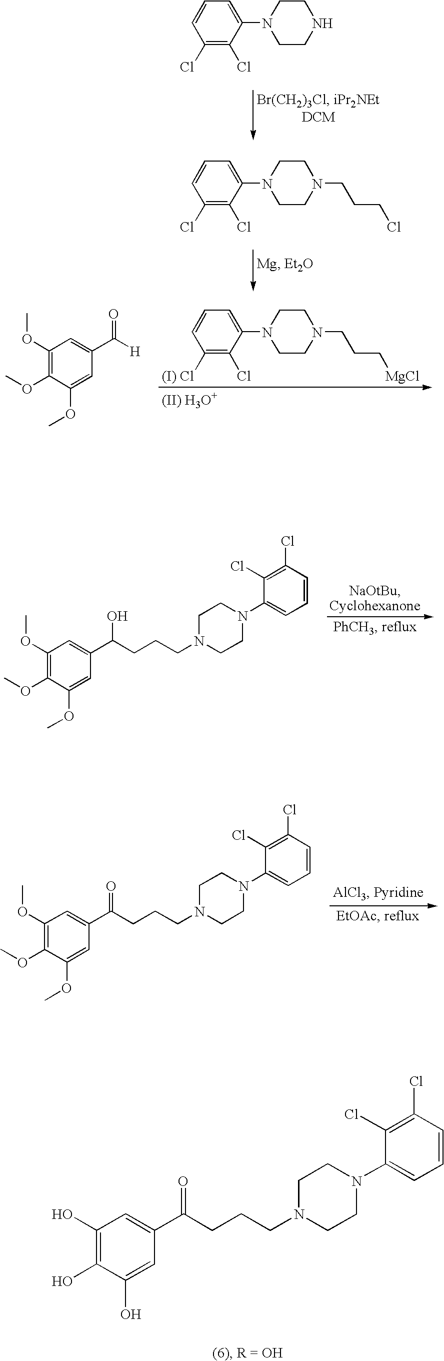
Synthesis of Compound (6), R=CN: 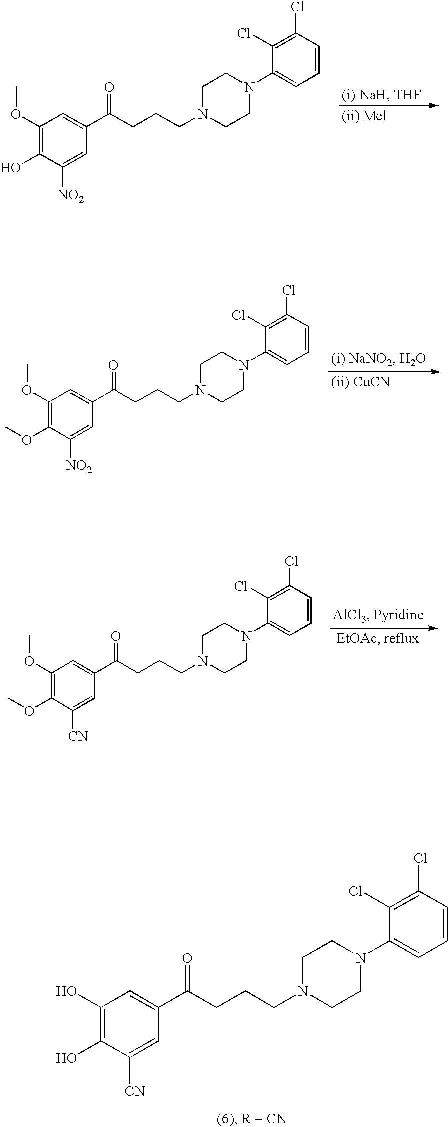
Synthesis of Amide Analogues of (5), (6) and (11), R=NO₂: 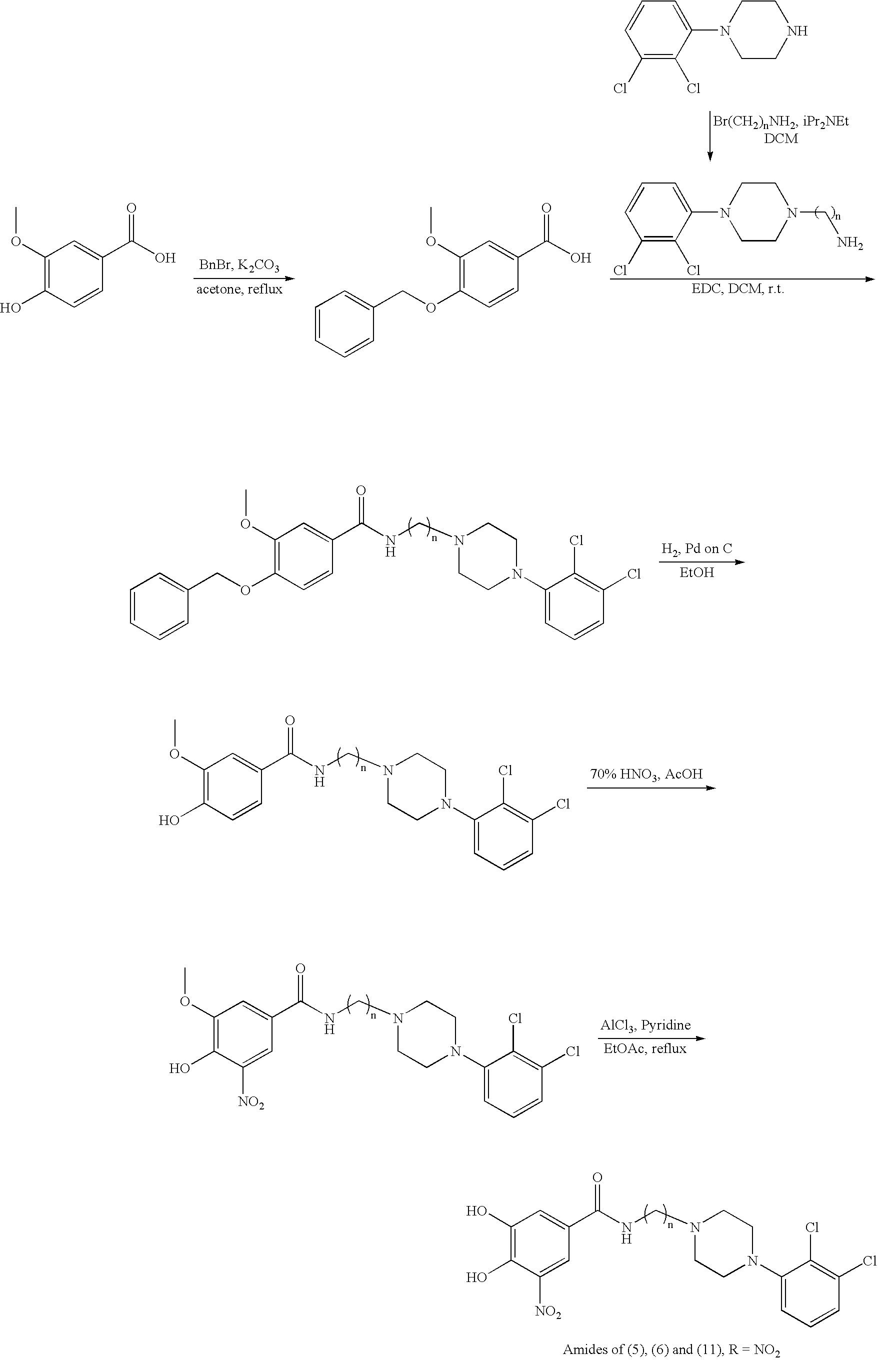
Synthesis of Amide Analogues of (5), (6) and (11), R=OH: 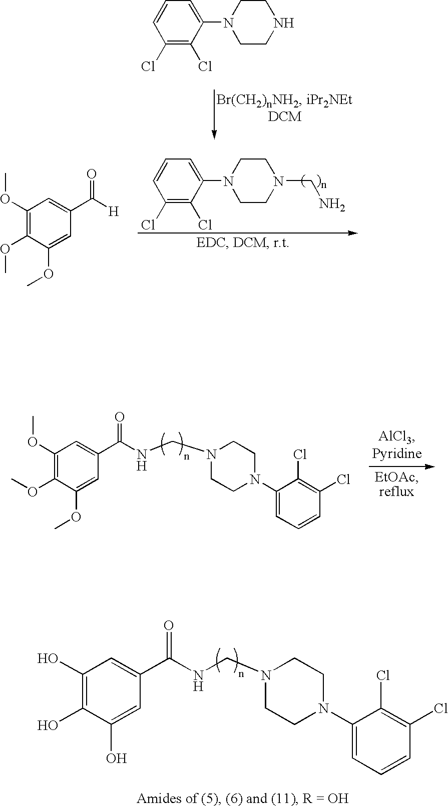
Synthesis of Amide Analogues of (5), (6) and (11), R=CN: 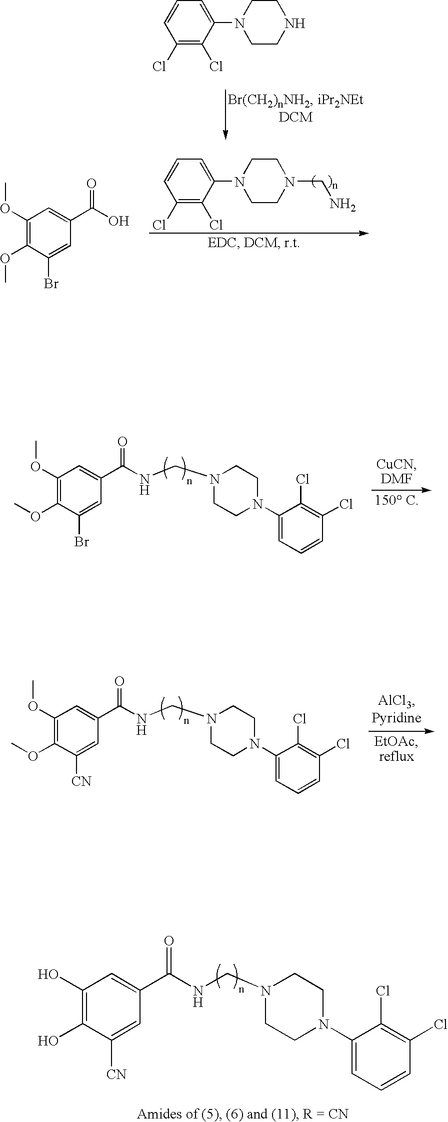
Synthesis of Compound (8): 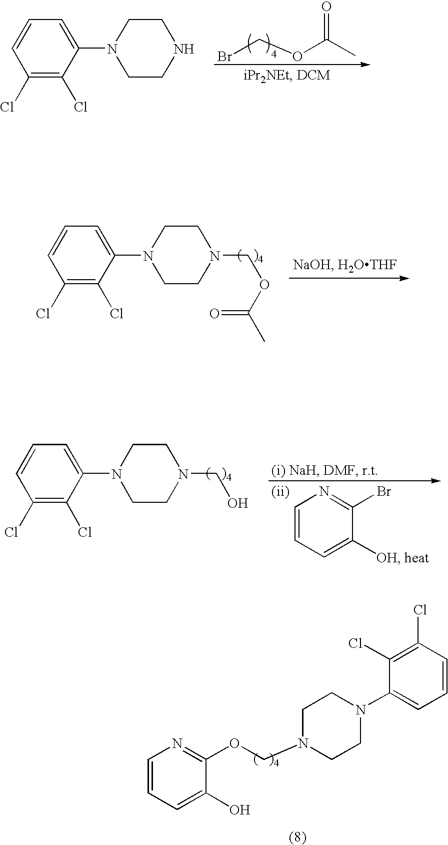
Compounds with various lengths of linker can be synthesized as shown: 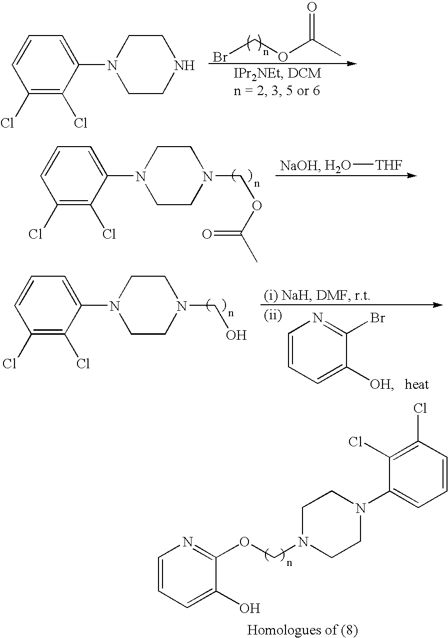
Synthesis of Compound (9): 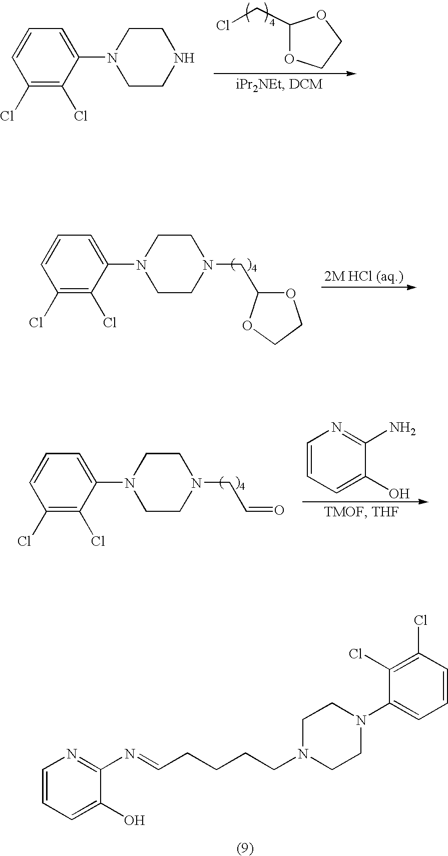
Compounds with various lengths of linker can be synthesized as shown: 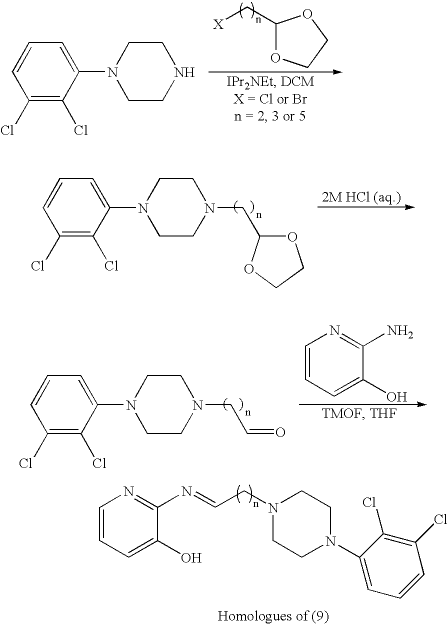
Analogues more similar in structure to CGP 28104 can be synthesized: 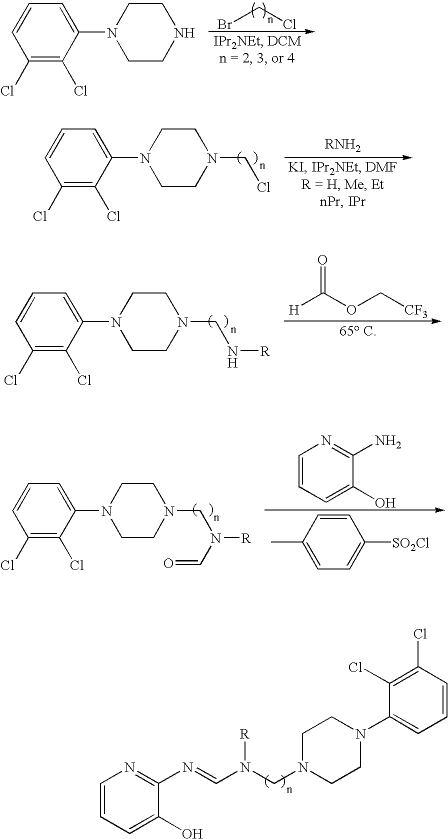
Synthesis of Compound (11), R=NO₂: 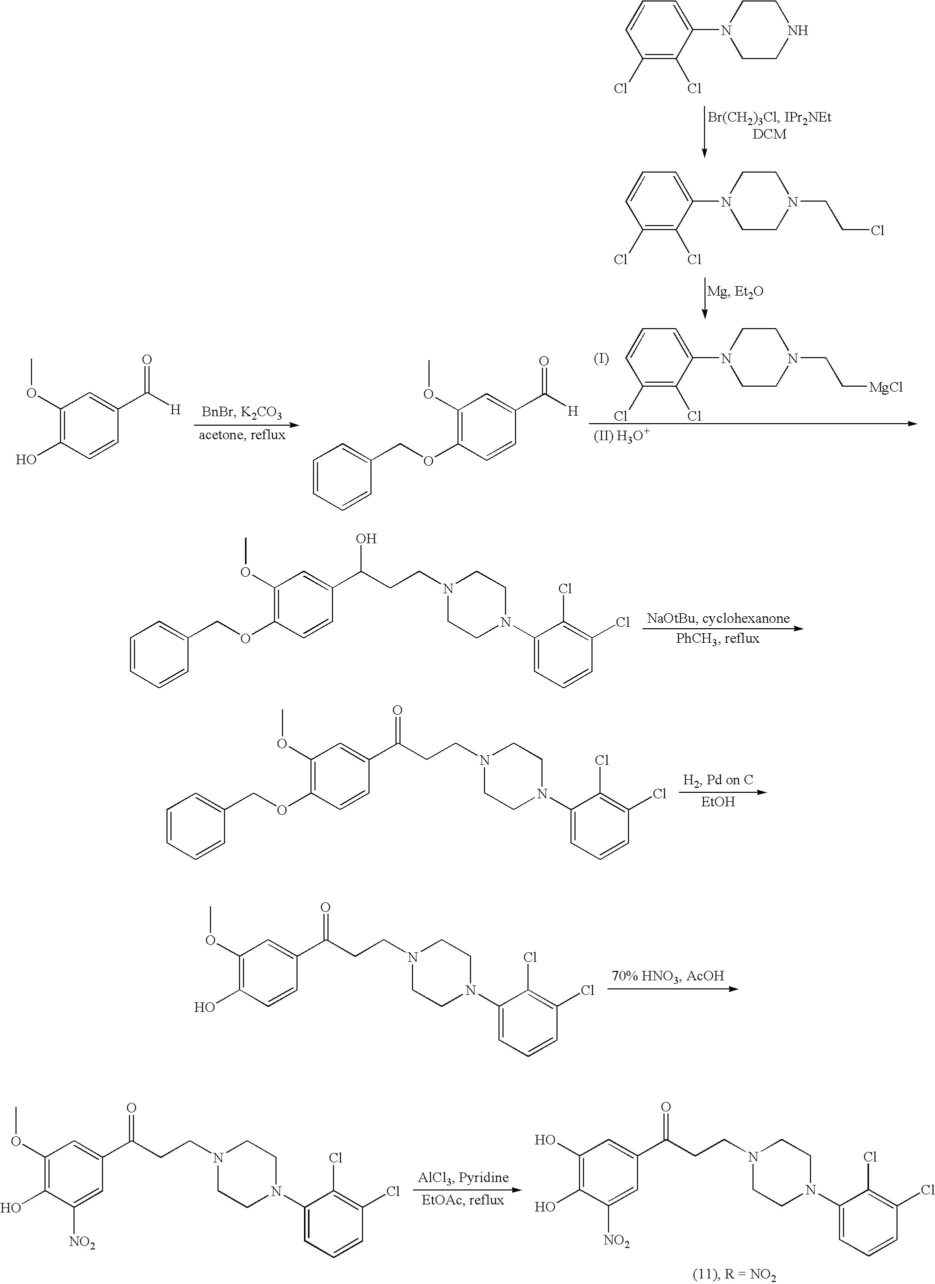
Synthesis of Compound (11), R=OH: 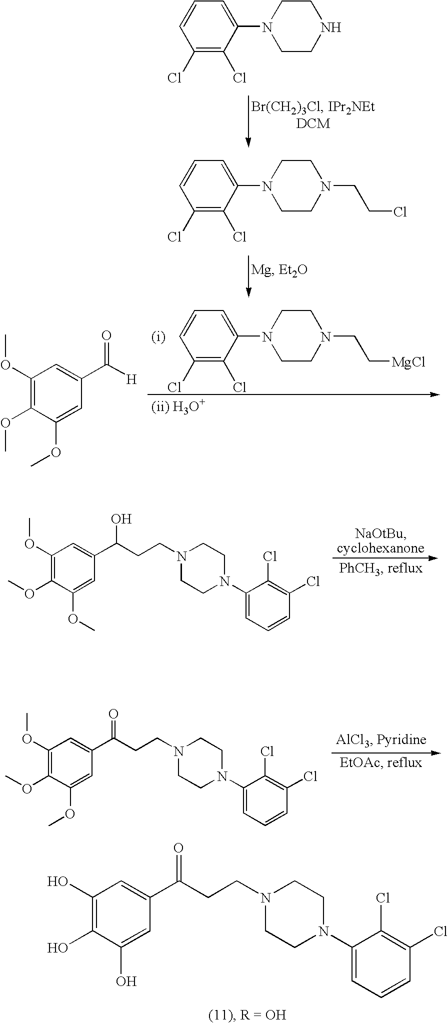
Synthesis of Compound (11), R=CN: 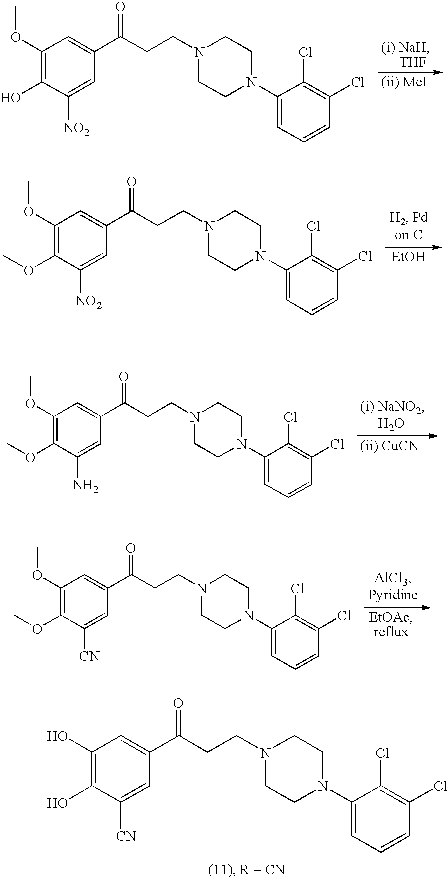
Synthesis of Compound (14), R=NO₂: 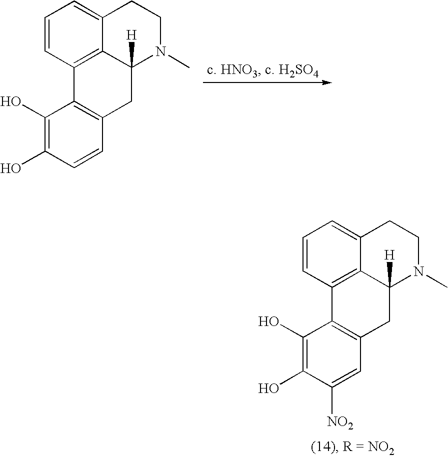
Synthesis of Compound (14), R=CN: 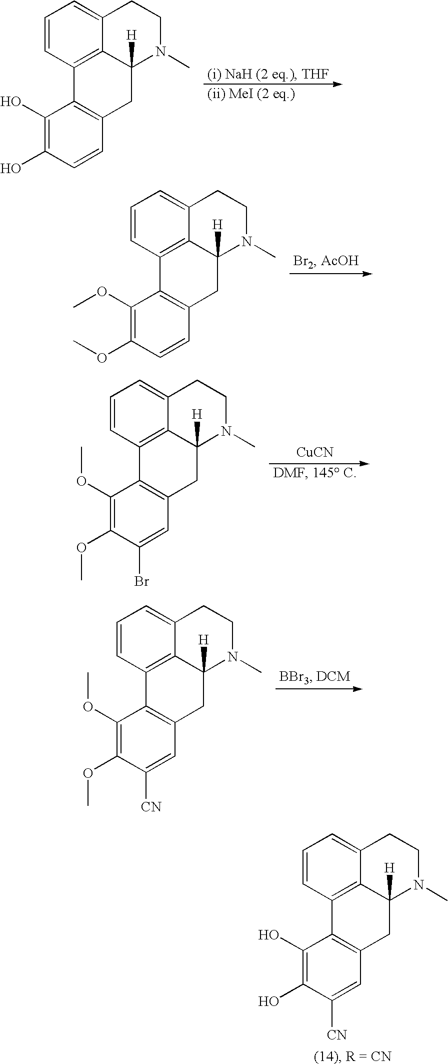
Synthesis of Compound (14), R=OH: 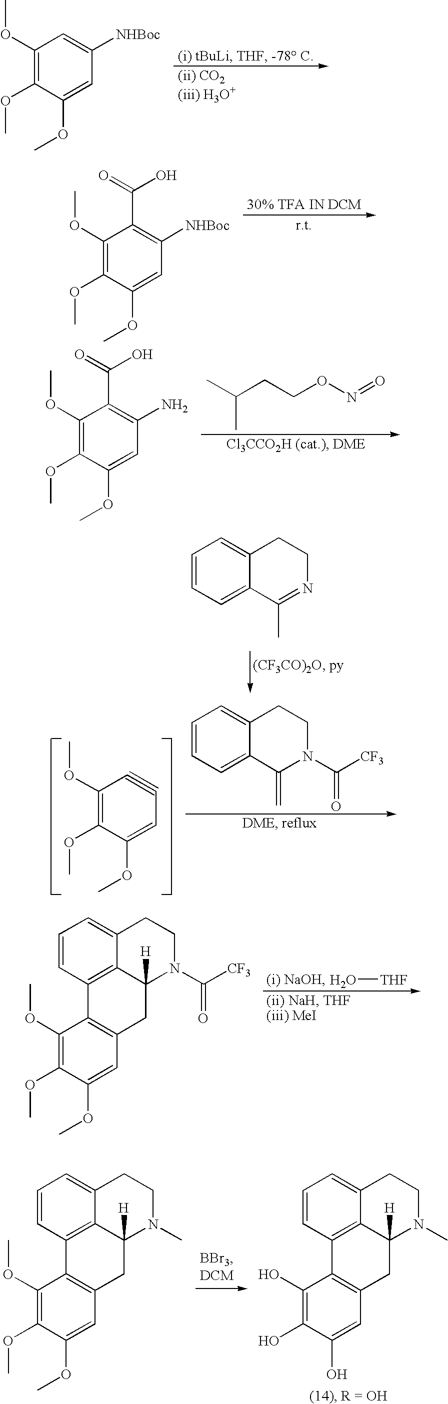
Synthesis of Compound (18), R=OH: 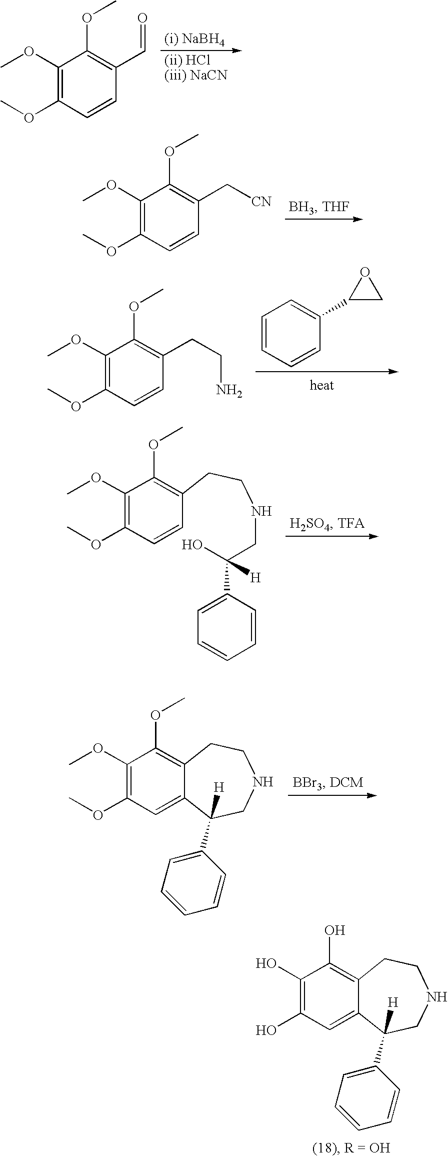
Synthesis of Compound (18), R=NO₂: 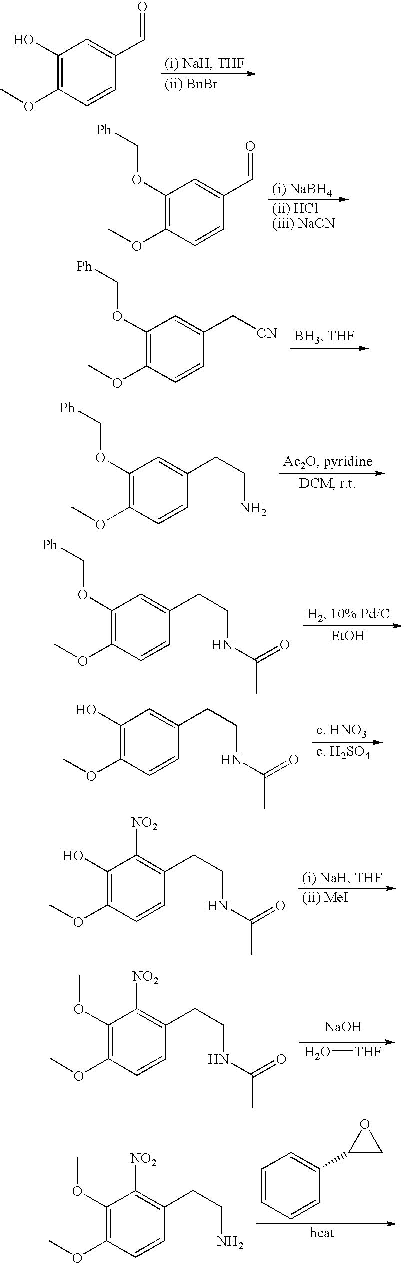 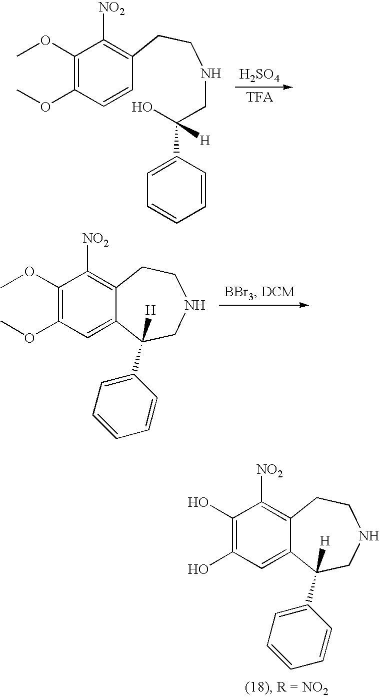
Synthesis of Compound (18), R=CN: 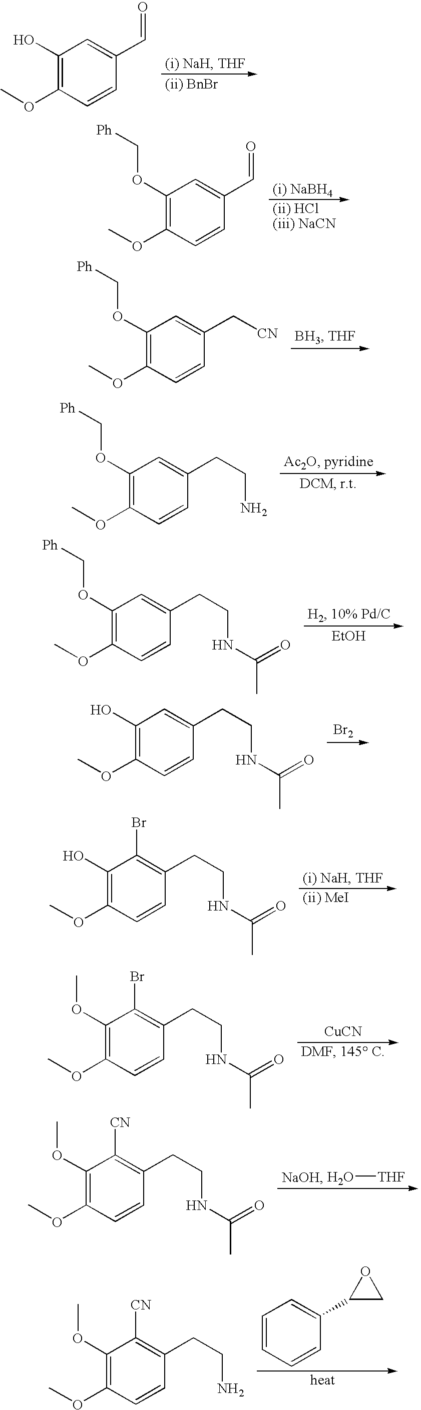 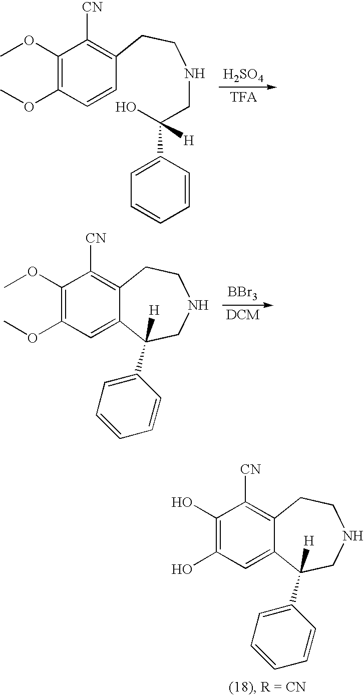
Synthesis of Compound (19), R=OH, NO₂ or CN: 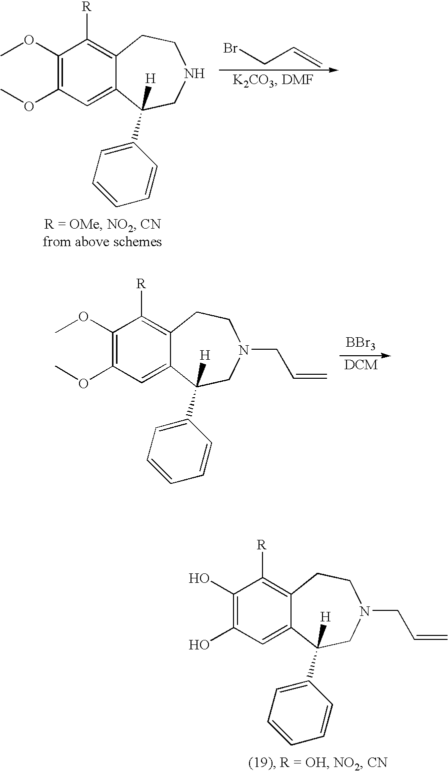
Synthesis of Compound (20), R=OH: 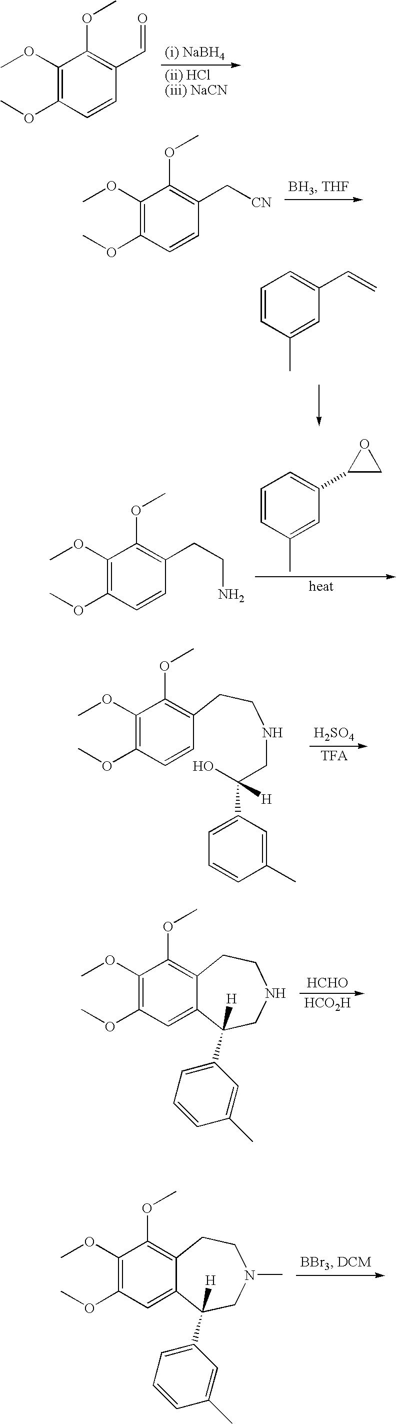 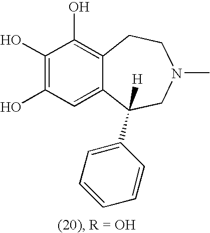
Synthesis of Compound (20), R=NO₂: 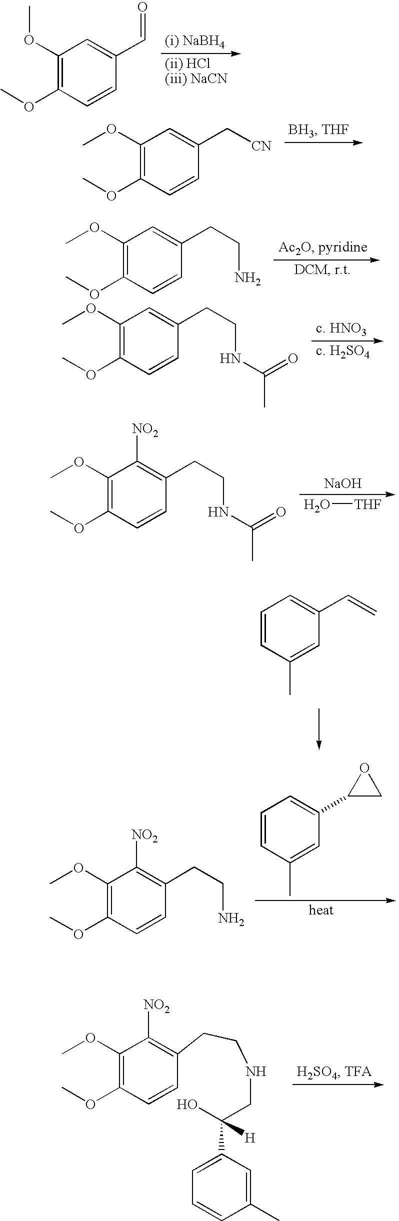 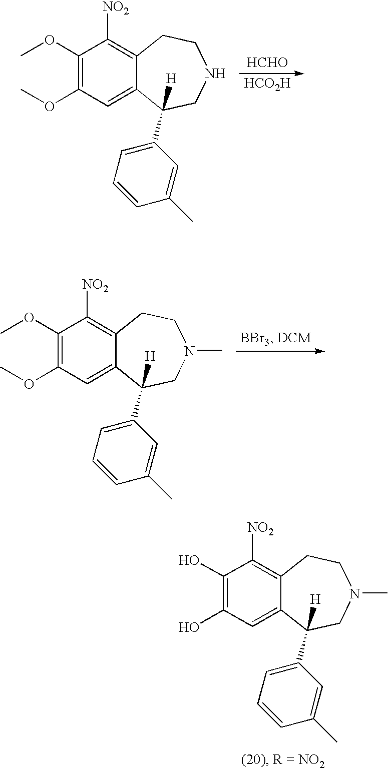
Synthesis of Compound (20), R=CN: 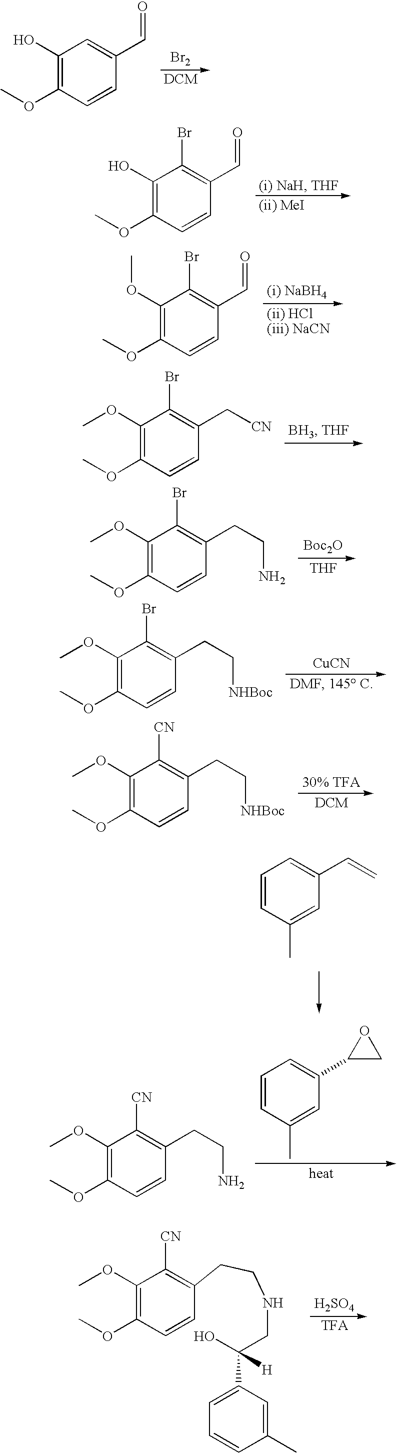 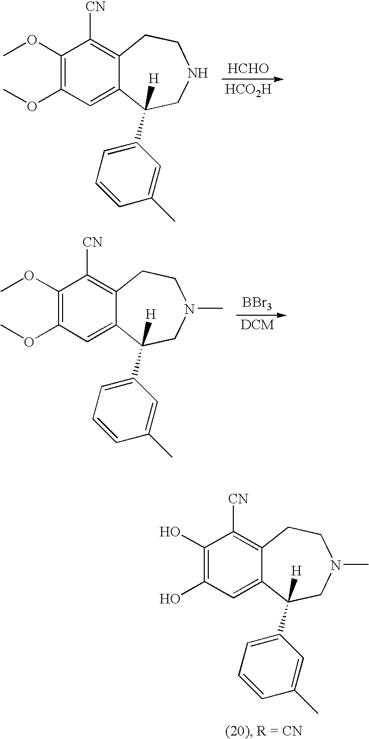
Synthesis of Compound (25), R=NO₂, R1=H: 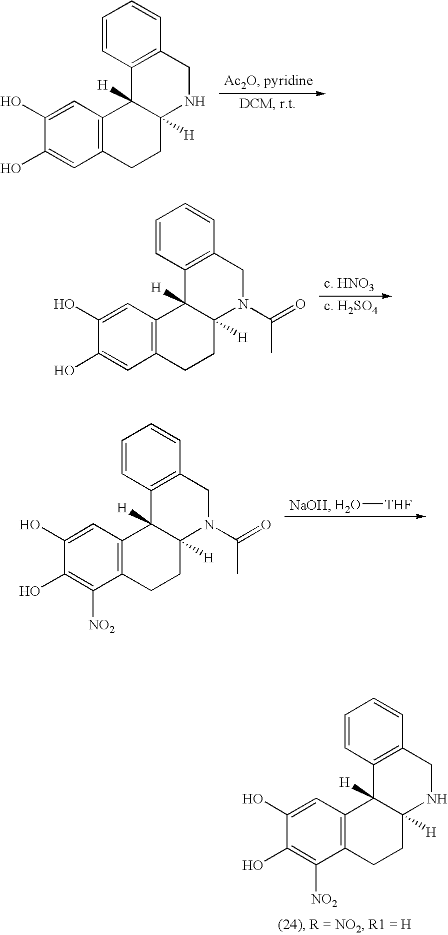
Synthesis of Compound (28), R=NO₂: 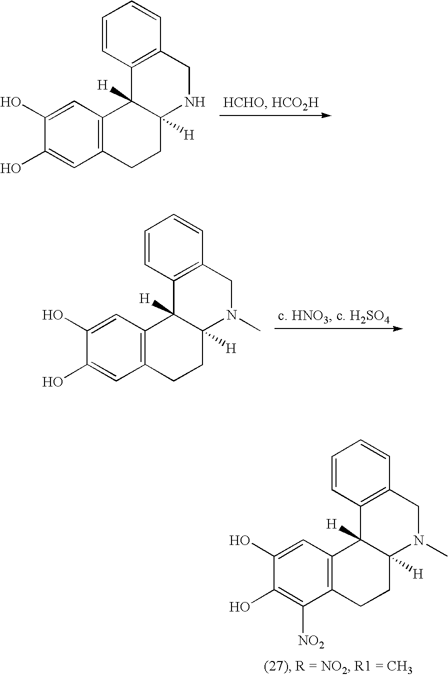
Synthesis of Compound (31), R=NO₂:

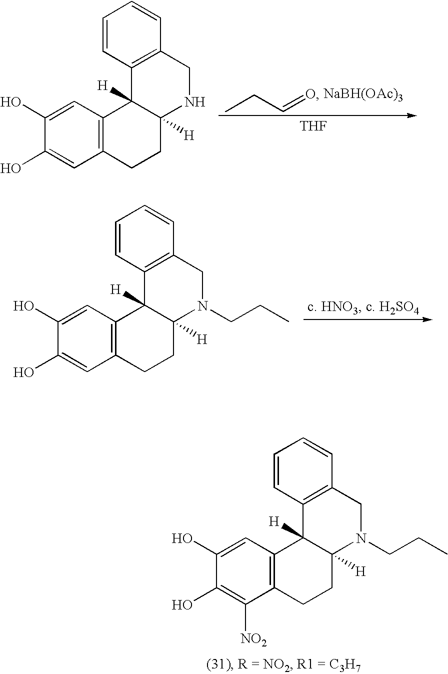

Compounds (24), R=OH, R1=H, (26), R=CN, R1=H, (27), R=OH, R1=CH₃, (29), R=CN, R1=CH₃, (30), R=OH, R1=n-C₃H₇, (32), R=CN, R1=n-C₃H₇ can be synthesized in a manner similar to that used for compounds (14), R=OH or CN above.

Compounds (33-41) can be synthesized in a manner similar to that used for compounds (24-32) above.
Synthesis of Compounds (43), (46) and (49), R=OH: 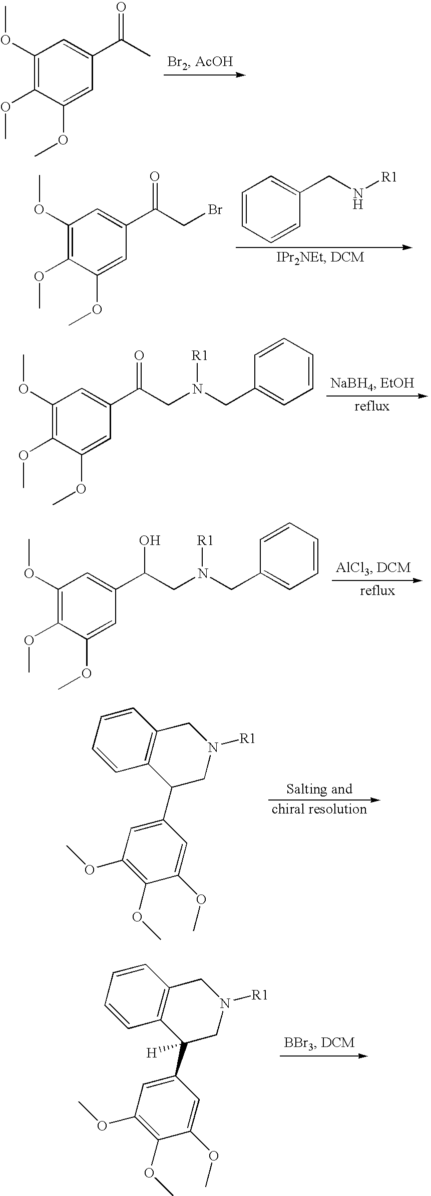 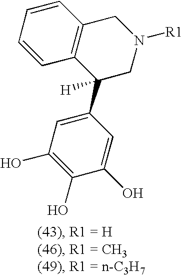
Synthesis of Compounds (44), (47) and (50), R=NO₂: 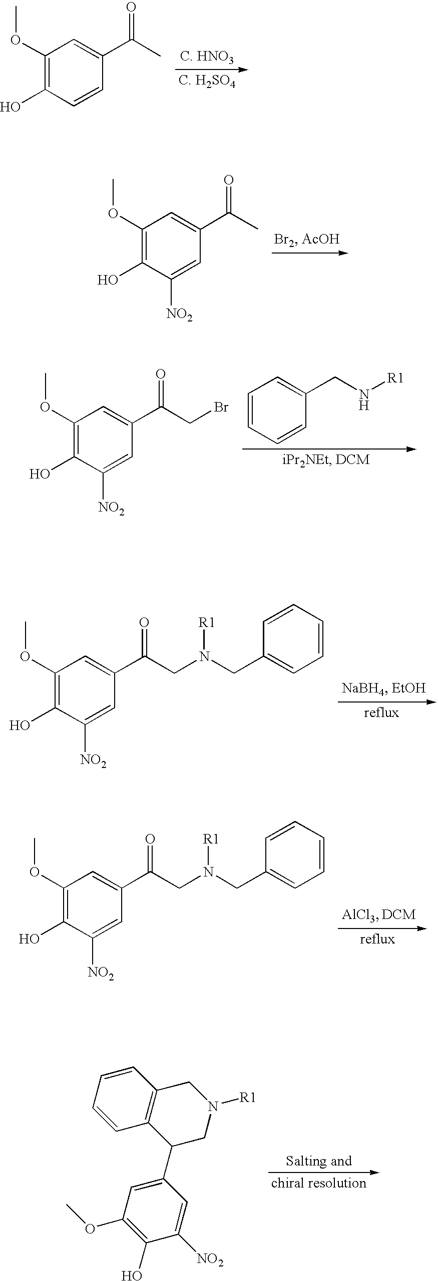 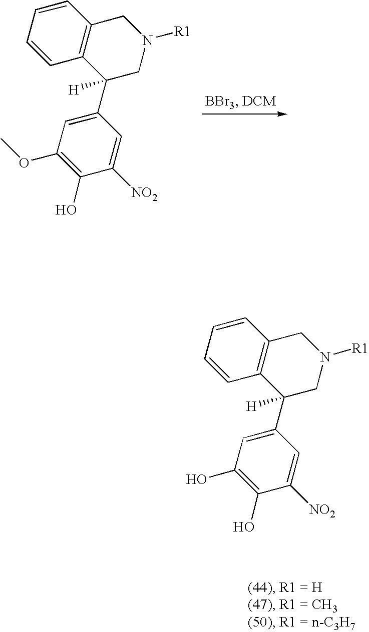
Synthesis of Compounds (45), (48) and (51), R=CN: 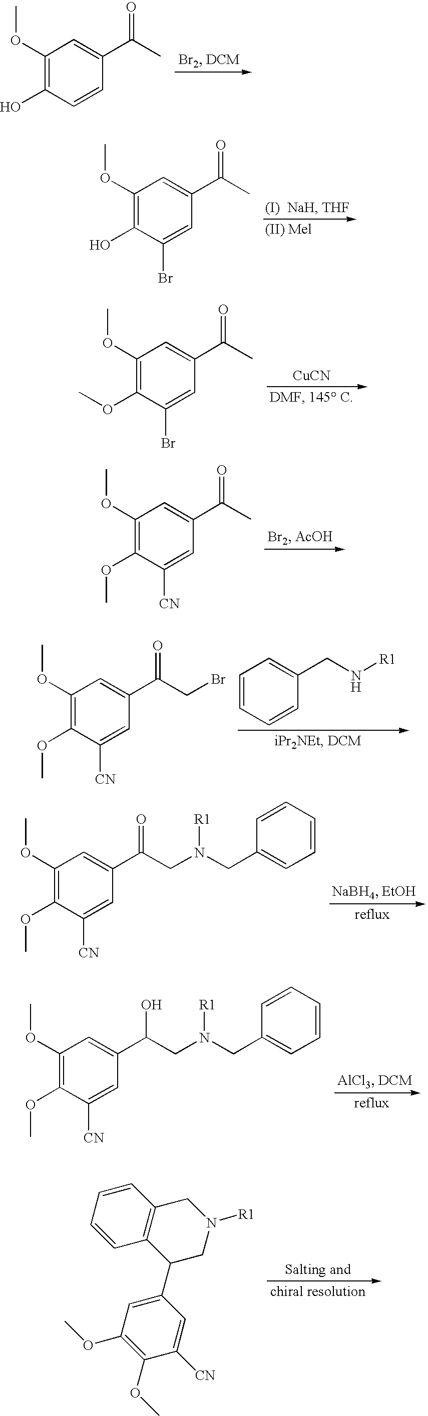 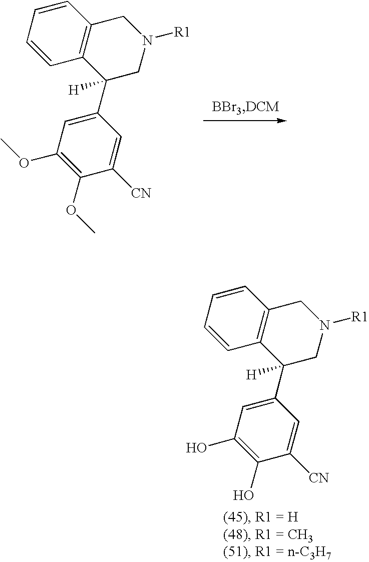
Synthesis of Compounds (52), (55) and (58), R=OH: 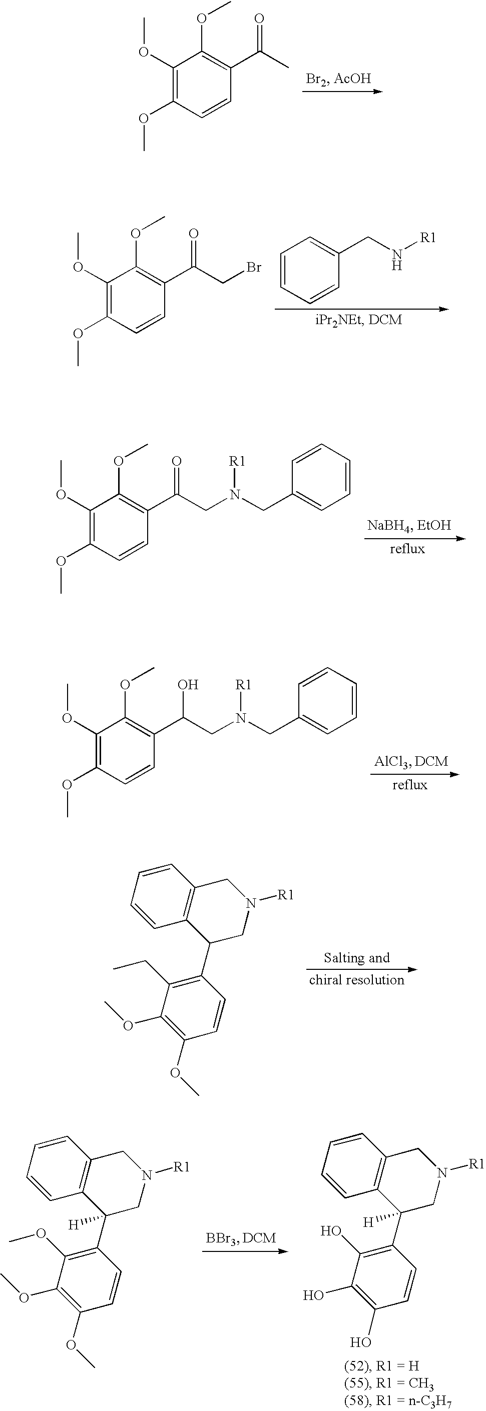
Synthesis of Compounds (53), (56) and (59), R=NO₂: 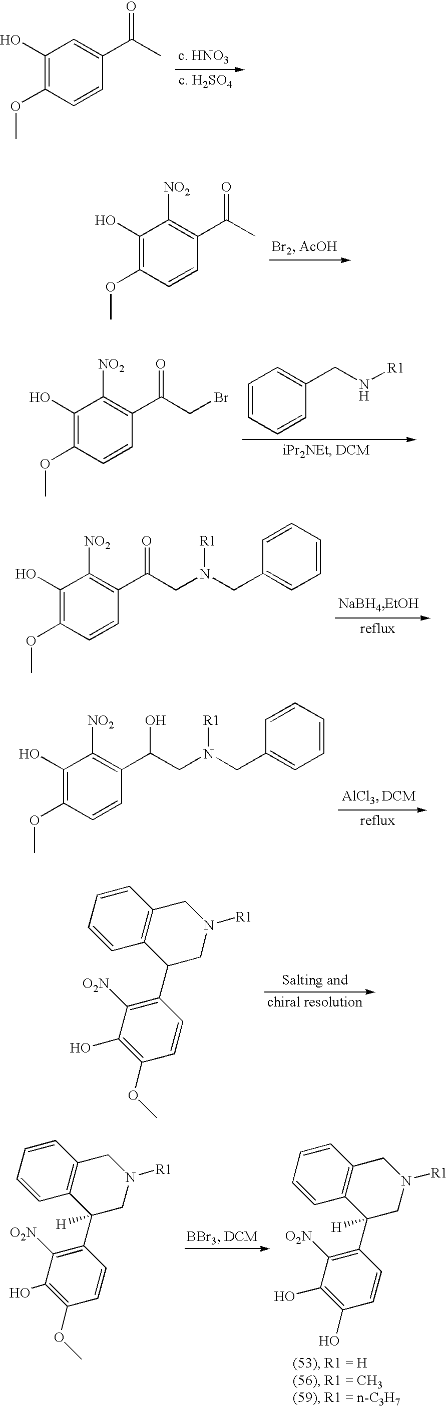
Synthesis of Compounds (54), (57) and (60), R=CN: 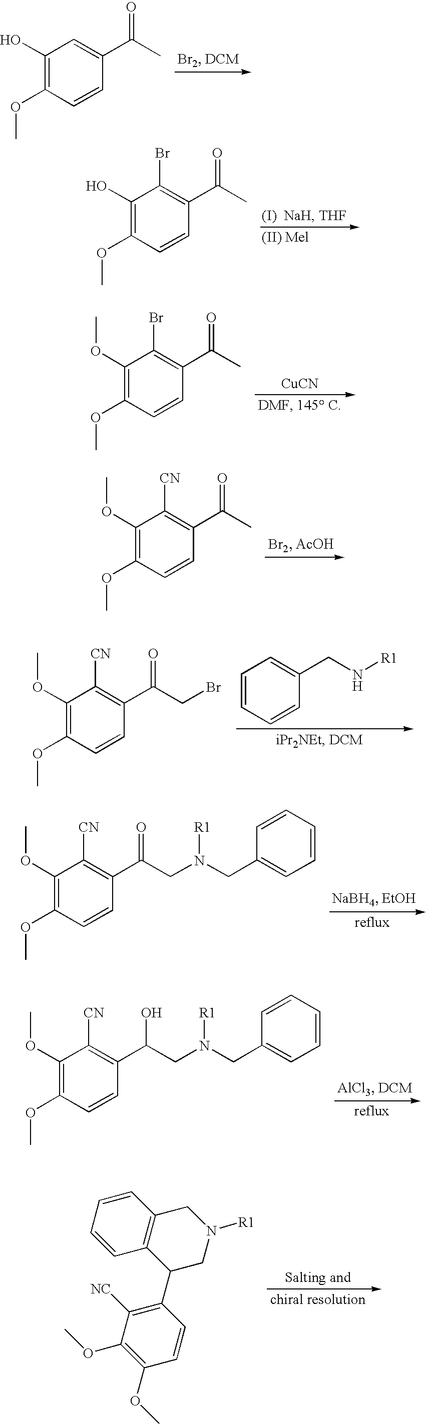 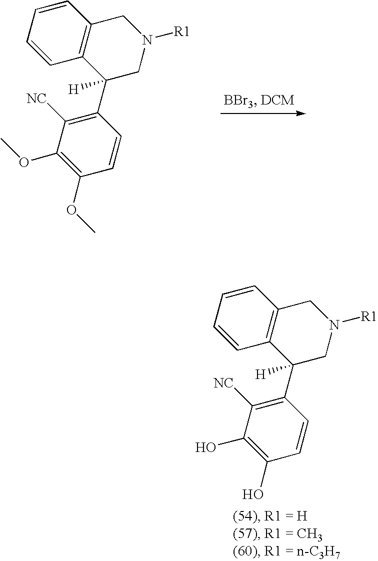
Compounds 62-79 can be prepared by replacing the phenyl ring with 2-thienyl, i.e.:
Replace 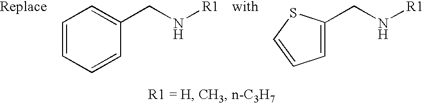
   For R1=H, the starting material is commercially available. For R1=CH₃ or n-C₃H₇, the starting materials can be synthesized as below: 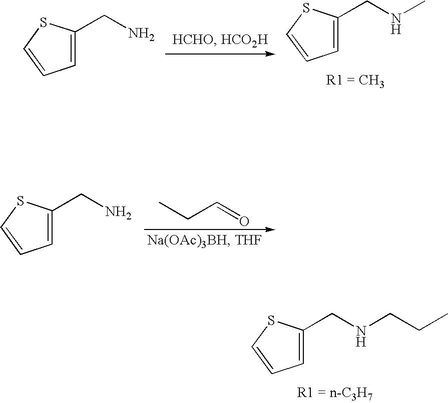
   Synthesis of Compounds (82), (85) and (88), R=NO₂: 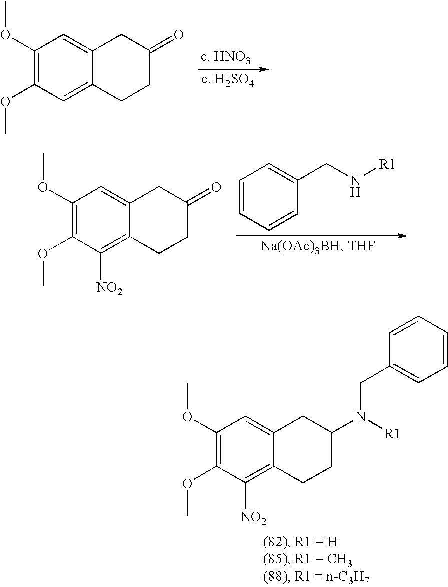
   Synthesis of Compounds (83), (86) and (89), R=CN: 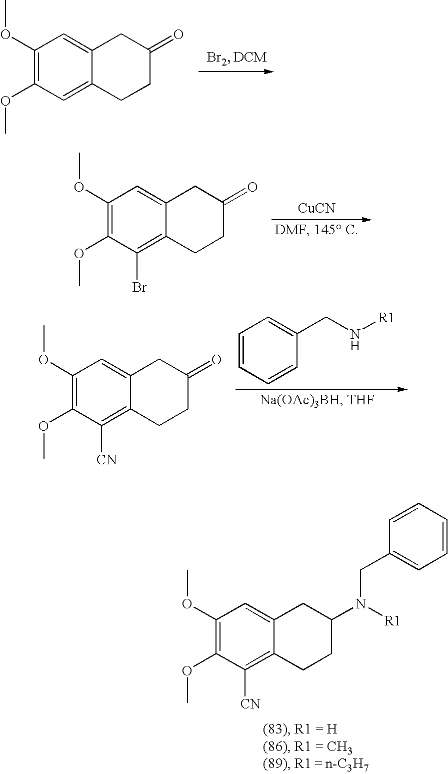
   Synthesis of Compounds (91), (94) and (97), R=NO₂: 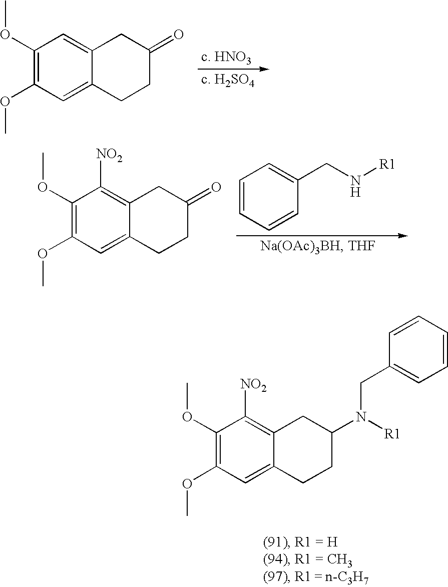
   Synthesis of Compounds (92), (95) and (98), R=CN: 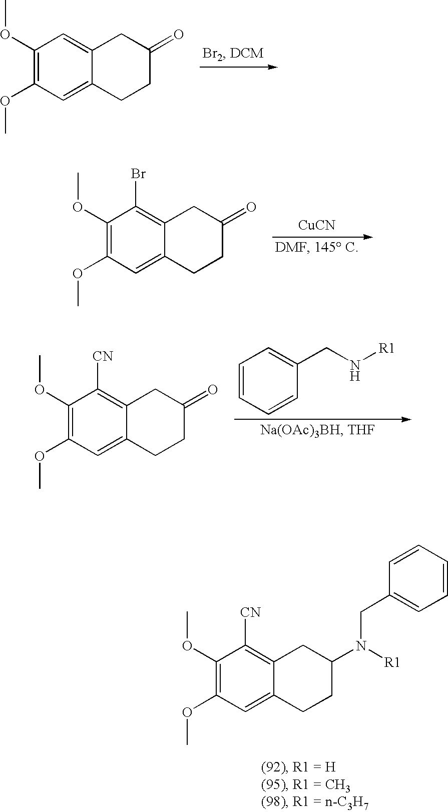
   Synthesis of Compound (100), R=OH: 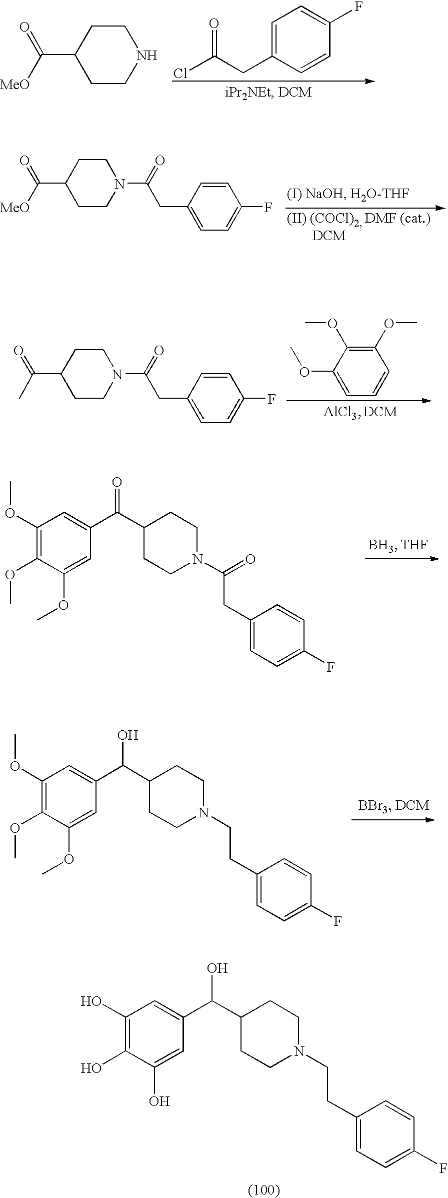
   Synthesis of Compound (101), R=NO₂: 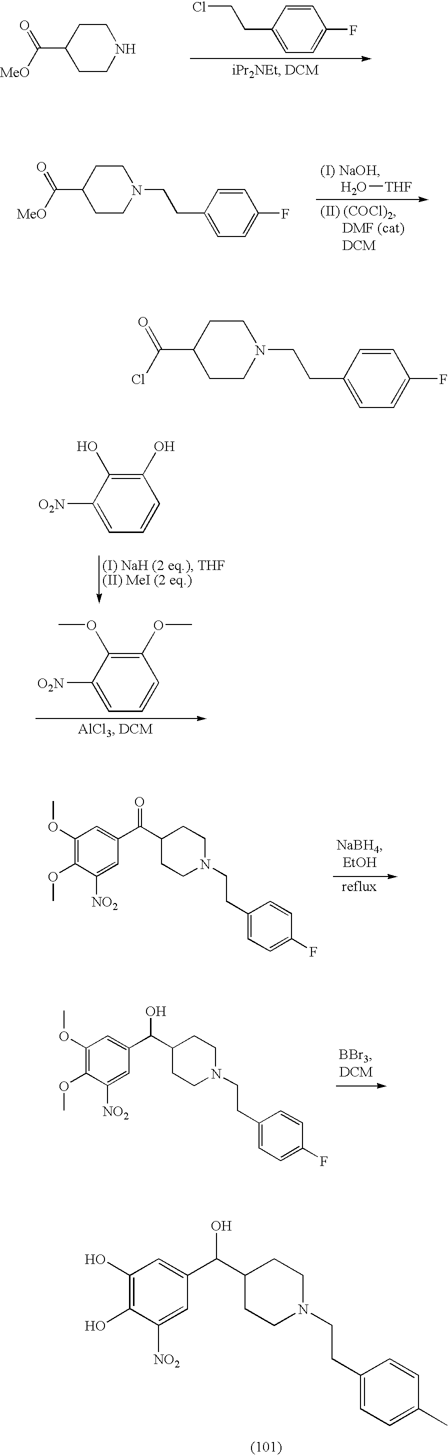
   Synthesis of Compound (102), R=CN: 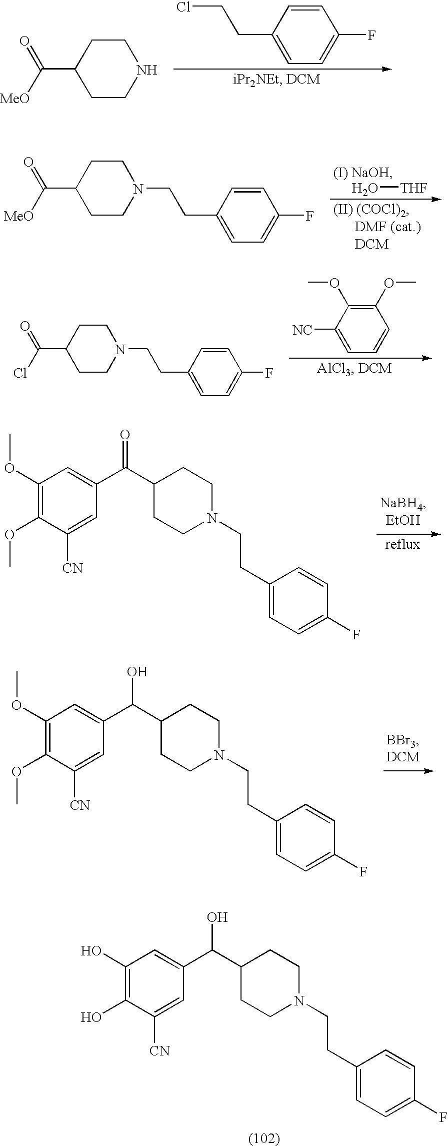
   Note: Entacapone (2) has the structure:

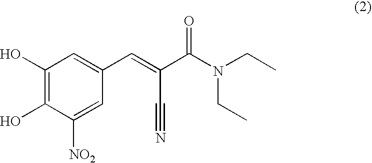

Compound (4), R=NO₂=compound (3)

Compound (12)=compound (6).

### EXAMPLE 11

### Synthesis of PGX 2000001 through 2000004

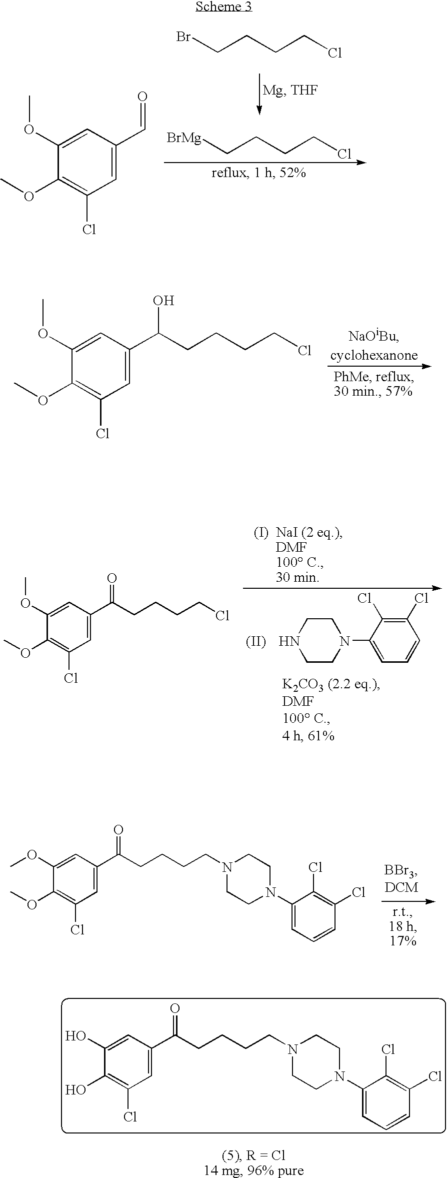

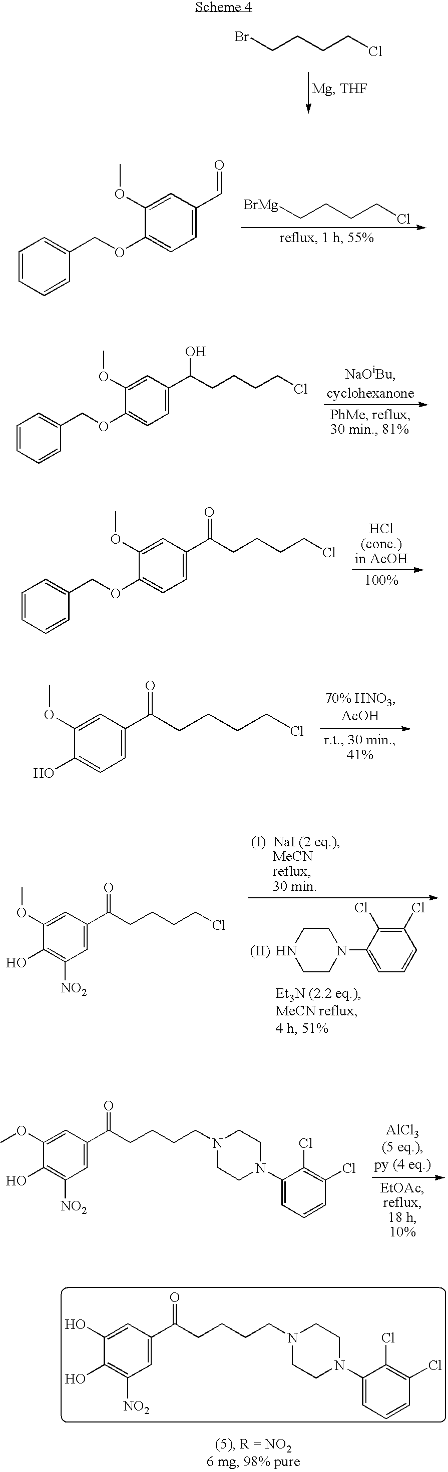

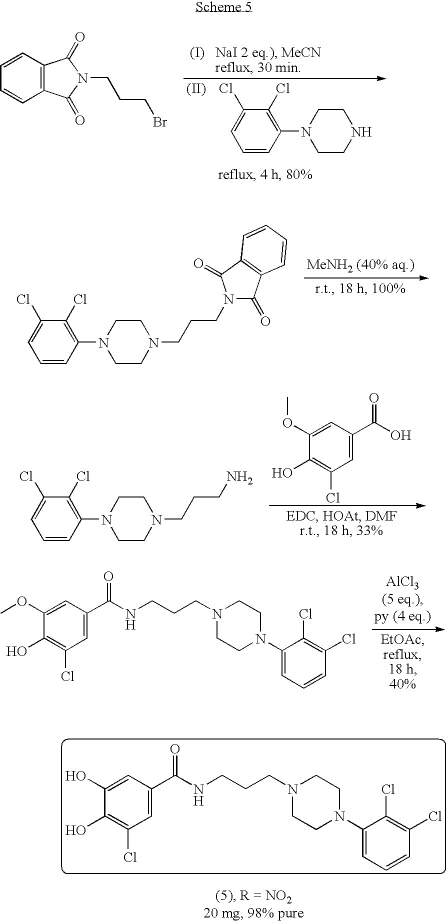

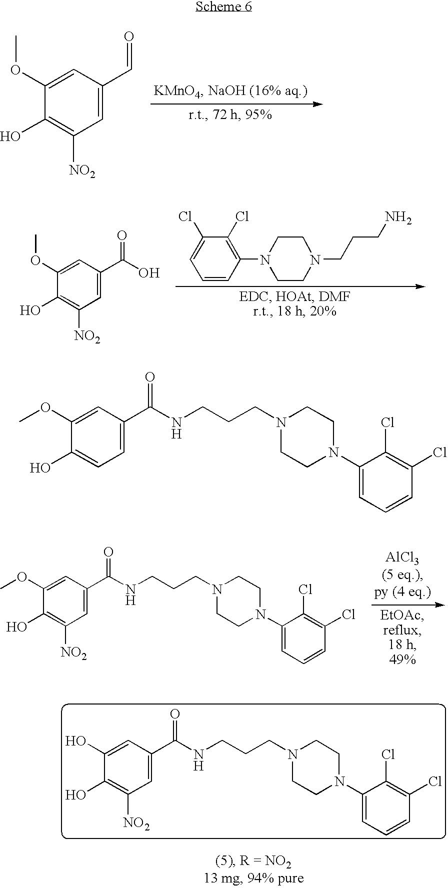

### EXAMPLE 12

### Method for Finding Evaluating Potential COMT Inhibitors with Dopamine D1 Agonist and/or D2 Partial Agonist or Antagonist Activity

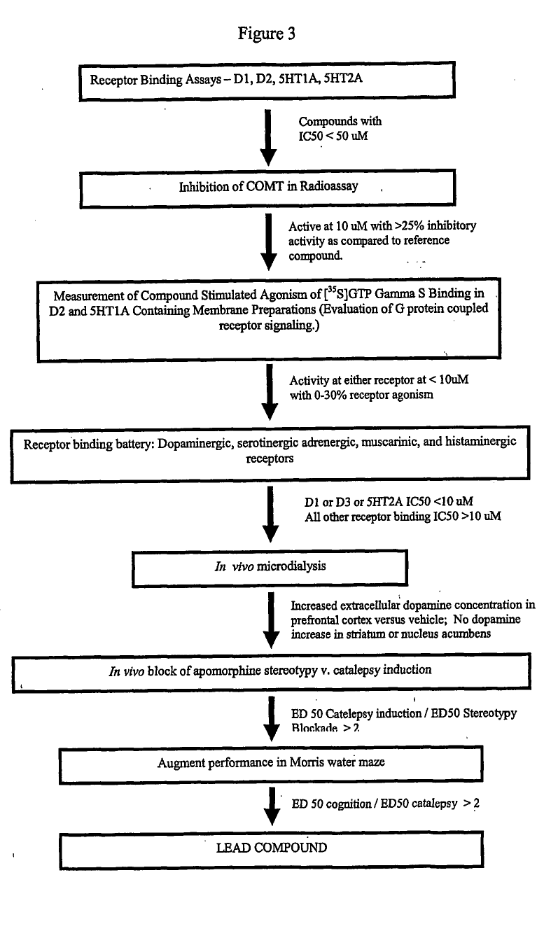

One objective of this invention is to use compounds for the treatment of schizophrenia or mild cognitive impairment that have been designed using rational design principles to act at several biologic sites relevant to the etiology of either disorder. As discussed above, these sites include, but are not limited to, the D1, D2, 5-HT1A and 5-HT2A receptors as well as the COMT enzyme. A second objective is to identify unique compounds (i.e. compounds not previously known to have psychoactive properties) that are capable of treating the negative and positive symptoms associated with schizophrenia. Testing the biochemical and functional activity of compounds to assure the correct mix of properties can be conducted using a combination of well-established methods. The outline for validating compounds is provided in FIG. 3.

Binding at Dopamine D1, D2, Serotonin 5-HT1A, and 5-HT2A Receptors

An initial step in compound evaluation involves the testing of the compounds ability to bind at receptors generally believed to be important for the functioning of candidate drugs. The form of receptor assay particularly amenable to the paradigm described here involves competitive displacement by the novel compound of the high-affinity binding by a pharmacologically active radiolabeled compound. For example, the potent binding affinity of [3H]sulpiride (affinity constant, or K_{D}=3.0 nM to D₂ receptors in the rat striatum) provides a basis for evaluating a test compound's potential activity at this receptor. Compounds having a high, ie., nM to uM, binding affinity have the effect of displacing sulipride at the D₂ receptor (Imafuku, J., Brain Res (1987) 402, 331-8). Similar binding assays are well-known in the art for the D1 receptor (Billard, W. et al., Life Sci (1984) 35, 1885-93), the 5-HT1A receptor (Hoyer, D. et al., Eur J Pharmacol (1985) 118, 13-23) and the 5-HT2A receptor (Leysen, J. E. et al., Mol Pharmacol (1982) 21, 301-14). Generally, compounds maybe pre-screened at higher concentrations (e.g, 50 uM) to evaluate activity of the compound. Subsequently, IC50 curves are performed using suitable dilutions. Compounds of particular interest will have IC50 values of 10 uM or less (FIG. 3).

Catechol-O-Methyl Transferase Inhibition

A further step in evaluating the compound for efficacy in treating schizophrenia involves evaluation of its ability to inhibit catechol-o-methyl transferase (COMT). The standard reaction involves the transfer of a radiolabeled methyl group contained in S-adenosyl methionine to a catechol moiety on the substrate molecule. Several analytic methods exist for the quantitation of COMT activity. These include the monitoring of products and reactants by UV absorbance or the electrochemical detection of o-methylated products after HPLC separation. A simple form of the assay involves quantitating radioactive methylation of the catechol substrate in which ³H or ¹⁴C labeled S-adenosyl methionine is employed as a donor molecule and incorporated by substrate (Zurcher, G. et al., J Neurochem (1982) 38, 191-5). As with the receptor binding evaluation described above, higher concentrations of compound (ie., 10-50 uM) are initially tested and IC50 curves determined later for compounds of that show inhibitory activity at the 10 or 50 uM concentration.

COMT enzyme that is suitable for use in assays is available commercially (Sigma Chemical Co., St. Louis, Mo.) as a preparation from rat membranes, but also may be isolated from other sources using techniques well-known in the art. The enzyme exists in a soluble and membrane bound form with the membrane bound form predominating in the brain. The proteins are coded for by the same gene, however, they have alternate start sites and thus localization within the cell. Since the methyl transferase activity is present in both forms of the enzyme, either may be utilized in evaluating the potential of antipsychotics agents. It is envisioned that human valine/methionine allelic variants of the enzyme will be used for evaluation of the drug's potential efficacy, as this single amino acid substitution determines thermal stability and thus activity of the COMT protein product.

G-Protein Coupled Receptor Signaling

Both dopamine and 5-hydoxythyamine receptors are part of the G-protein coupled family of receptors (GPCR). Since the mediation of signaling by dopamine and 5HT receptors occurs through this second-messenger system, the effect of potential antipsychotics agents may be measured through analysis of G-protein coupled events. Upon activation, GPCRs exert many of their cellular actions through promotion of guanine nucleotide exchange on the Gα-subunit of heterotrimeric G-proteins to release free Gα-GTP and βγ subumits. In membrane preparations, GTP can be substituted by the non-hydrolyzable form, ³⁵S-labeled guanosine 5′-O-(3-thio)triphospphate (³⁵S-GTPγS). In the presence of agonist stimulation, a stable ³⁵S-GTPγS-Gα complex will form and accumulate due to its resistance to hydrolysis. (Dowling, M. R. et al., Receptor Signal Transduction Protocols (2004) 197-206). For example, membranes prepared from cells that contain dopamine D₂ receptors, either through their natural presence of following their stable transfection into the cell line of interest, can be prepared by routine tissue homogenization and can be stored frozen until use. Exposing the membranes to full agonist ligands, such as quinpriole or dopamine itself defines maximal levels of ³⁵S-GTP binding, by which experimental compounds can be evaluated. A partial agonist, one goal of the presently proposed compounds, will produce less than the maximal signal produced by dopamine, and an antagonist will not alter ³⁵S-GTP binding per se, but will block the increase in ³⁵S-GTP binding produced by full or partial D₂ agonist ligands (Dowling, M. R., Nahorski, S. R., Receptor Signal Transduction Protocols (2004) 197-206)

Receptor Binding Battery

The specificity of the proposed compounds for their binding to dopamine and serotonin receptors can be measured by radioligand binding methods described above. The demonstration of selectivity of their binding to these sites requires similar radioligand binding methods, typical and well-known to those skilled in the arts, applied to the other common GPCR, ion channel, and other membrane-bound receptors. Such an approach is standard in the arts and routinely applied by commercial services such as NovaScreen, Inc. It is expected that the molecules invented here will show less significant binding to non-dopamine, non-serotonin receptors.

In Vivo Brain Microdialysis

In vivo brain microdialysis is used to continuously monitor basal monoamine concentrations in brain extracellular fluid (bECF) of freely moving rats before and after vehicle or drug treatment. Stereotaxic surgery will be conducted under aseptic conditions to implant 2 guide cannulae and microdialysis probes in the medial prefrontal cortex and the neostriatum of anesthetized rats.

As described (Jordan, S. et al., Eur J Pharmacol (2004) 483, 45-53), rats are anesthetized and shaved on the top of the head. The shaven area is sterilized with betadine, and a sterile gel “artificial tears”) is applied to each cornea to prevent drying of the eyes during surgery. The anesthetized animal is positioned in a stereotaxic frame upon a thermal blanket set at 37 degrees C. or a Glycol pack warmed to this temperature. A midline incision is made on the shaved surface of the head and the tissue is retracted to expose the skull, which is subsequently cleaned with sterile Q-tips. Two or 3 stainless steel surgical screws are screwed into the and away from the two cannula hole sites. A burr hole (1 mm diameter) is drilled through the skull at a stereotaxic coordinate (3.2 rostral to bregma and 0.6 mm lateral to the sagittal suture) directly above the medial prefrontal cortex (MPFC). Another hole is drilled at the rostral-caudal level of Bregma and 3 mm lateral to the midline, on the opposite side as the frontal cortex site. Dura is pierced with a 25-gauge needle and a microdialysis probe is lowered over 2 min to 5.3 mm below dura (frontal cortex) or 6.3 mm below dura (striatum).

The cannulae and probes are anchored to the skull surface using dental acrylic. The retracted tissue is repositioned around the base of the cannula and sealed with Vetbond glue. The animal is maintained during recovery in its home cage under a warm lamp, prior to its return to the animal facility. The animal will be allowed to recover for 24 hr±2 hr before the first microdialysate is collected.

A ‘Raturn’ system (Bioanalytical Systems, Indiana) will be used to house each rat during each microdialysis procedure. This apparatus consists of a Perspex observation arena (50 cm wide×100 cm high) with a rotating base plate. Infrared detection senses rat movements inside the bowl and this leads to a compensatory rotation of the bowl, thus preventing tangling of the probe cannula

The cannula and probe will be continuously perfused at 1 ul/min with an artificial ECF solution (Ringers solution) and the microdialysate will be collected every 30-min for 3 hr before and 4 hr after oral vehicle or drug dosing.

HPLC with electrochemical detection is be used to quantitate monoamines, particularly dopamine, norepinephrine (if possible), and serotonin in each 30 ul microdialysate.

The brain of some sacrificed animals is removed to verify the accuracy of cannula placements in the brain, and to determine if either drug regimen results in changes at the biochemical level. Trunk blood is used to potentially measure aripiprazole and tolcapone concentrations in serum. All data are evaluated statistically using ANOVA followed by Dunnett's t test for individual changes

Block of Apomorphine Sterotypy Versus Catalepsy

The preclinical efficacy and side effect liabilities of the drugs invented here can be evaluated in vivo by their potencies for, respectively, of inhibiting apomorphine-induced stereotyped behavior, an animal model for positive symptom treatment via D₂ antagonism (Hirose, T. et al., J. Psychopharmacol (2004) In Press), and their ability to induce catalepsy is an animal model for the induction of EPS (Hirose, T. Uwahodo, Y., J. Psychopharmacol (2004) In Press). The ratio of the ED50 for the drug to inhibit apomorphine-induced stereotypyed behavior relative to catalepsy induction is a measure of therapeutic ratio, or relative efficacy versus side effect potential.

Augment Performance in Morris Water Maze

The ability of the compounds described here to augment cognitive functions can be assessed with the Morris water maze, using apparati and experimental procedures similar to those described (Morris, R., J Neurosci Methods (1984) 11, 47-60, Tottori, K. et al., J Pharmacol Exp Ther (2002) 301, 249-57). The apparatus is a white circular pool (130 cm in diameter, 40 cm in height) filled to a depth of 30 cm with 24° C. water that is made opaque by the addition of non-toxic white dye. The pool is divided into 4 compass quadrants (north, south, east, and west). Diverse visual stimuli are permanently located on the walls beyond the tank in each of the 4 quadrants. A circular goal platform 10 cm in diameter is hidden 1 cm below the water level in the middle of one of the quadrants.

The continuous location of each swimming male F344 rat (150-245 g) from the start point to the goal is recorded by a video camera connected to a visual position sensor placed 1.25 m above the, surface of the water. Data from the sensor is analyzed on a PC. For the acquisition of spatial learning, each animal underwent a block of 4 trials/day for several days. Before the first trial, the rat is placed on the hidden platform for 30 s by the investigator. Each trial consisted of placing the rat in the water facing the wall of the pool at one of the randomly selected four starting locations around the pool perimeter. Each rat is allowed a maximum of 60 s to find the hidden platform and remain on it for 30 s. If a rat failed to find the platform within 60 s, the rat is placed on the platform for 30 s by the investigator. The time and distance required to find the hidden platform during these 4 acquisition trials are averaged

A block of 4 qualifying trials is conducted the day after the end of the learning acquisition period. Only those rats that found the hidden platform between 5-10 s in time and between 100-200 cm in distance are used in subsequent experiments conducted in the afternoon of the same day. One of the compounds invented here is administered orally and 60 min later the rats undergo a block of 4 trials.

D) Statistical Analysis

AllAll data are evaluated statistically using ANOVA followed by Dunnet's t test for individual changes

Dosing and Dosage Forms:

Compounds claimed in this invention are administered as an ‘effective amount’ in the form of tablets, capsules, suppositories or intravenous or intramuscular injections. The ‘effective amount’ of compound is that amount that will prevent occurrence of negative and/or positive symptoms of schizophrenia and/or enhance the cognitive function of the patient. Patients who will benefit from this invention include those with schizophrenia, schizoaffective disorder, bipolar disorder and cognitive deficits associated with Alzheimer's disease and age-related mild cognitive impairment. The effective dose can vary, depending on the severity of the symptoms and the manner in which the compound is administered. Dosage ranges are 1 to 500 mg up to 4 times per day. The preferred embodiment is 10-100 mg administered no more than 2 times per day by the oral route.

### REFERENCES CITED

Altar, C. A., Martin, A., and Thurkauf, A. “Antipsychotic Agents”. In: Burger's Medicinal Chemistry and Drug Discovery (6^{th} Edition), Vol. 6, Nervous System Agents 2003 (In press).
Altar, C. A., Wasley, A. M., Neale, R. and Stone, G. (1986) Typical and atypical neuroleptic binding at D2 and S2 receptors: An autoradiographic analysis in rat brain. Brain Res. Bull. 16: 517-525
Brewster, W. K., Nichols, D. E., Riggs, R. M., Mottola, D. M., Lovenberg, T. W., Lewis, M. H. and Mailman, R. B. (1990) J. Med Chem 33, 1756-1764
de Paulis T. M-100907 (Aventis). Curr. Opin. Investig Drugs 2: 123-132 2001
Egan, M. F., Goldberg, T. E, Kolachana, B. S., Callicott, J. H., Mazzanti, C. M., Straubb, R. E., Goldman, D. and Weinberger, D. R. (2001) Effect of COMT Val 108/158Met genotype on frontal lobe function and risk for schizophrenia. Proc. Natl. Acad. Sci. 98:6917-6922.
Floresco S B, Phillips A G (2001) Delayed-dependent modulation of memory retrieval by infusion of a dopamine D1 agonist into the rat medial prefrontal cortex. Behav. Neurosci. 115: 934-939
Gasparini M, Fabrizio E, Bonifati V, and Meco G (1997). Cognitive improvement during tolcapone treatment in Parkinson's disease. J. Neural Transm. 104: 887-894
Granon S, Passetti F, Thomas K L, Dalley J W, Everitt B J, Robbins T W (2000). Enhanced and impaired attentional performance after infusion of D1 dopaminergic receptor agents into rat prefrontal cortex. J. Neurosci. 20: 1208-15
Hyman S. E., and Fenton, W. S. (2003) What are the right targets for psychopharmacology? Science 299: 350-351
Joober, R., Gauthier, J., Lal, S., Bloom, D., Lalonde, Pl, Rouleau, G., Benkelfat, C., and Labelle, A. (2002) Catechol O-methyltransferase Val 108/158Met gene variants associated with performance on the Wisconsin card sorting test. Arch Gen Psych. 59: 662-3.
Kane J and Ingenito G (2000) Activity of aripiprazole in psychotic disorders: comparison with haloperidol and placebo. Int J Neuropsychopharmacol 3(suppl 1):S124
Liljequist R. Haapalinna A, Ahlander M, Li Y H, and Mannisto P T (1997) Catechol-O-methyltransferase inhibitor, tolcapone has minor influence on performance in experimental memory model in rats. Behav. Brain Res. 82: 195-202
Lotta, T., Vidgren, J., Tilgmann, C., Ulmanen, I., Melen, K, Julkunene, I. and Taskinen, J. (1995) Kinetics of human soluble and membrane-bound COMT: a revised mechanism and description of the thermolabile variant of the enzyme. Biochem. 34: 4202-10.
Matsumoto, M. Weickert, C. S, Akil, M., Lipska, B. K. Hyde, T. M. Herman, M. M. Kleimnean, J. E. and Weinberger, D. R. (2003) Catechol O-methyltransferase mRNA expression in human and rat brain: evidence for a role in cortical neuronal function. Neurosci. 116: 127-137.
McDermed, J. D., Freeman, H. S., and Ferris, R. M., in Catecholamines: Basic and Clinical Frontiers; Usdin, E. Kopin. I. J., Barchas, J. eds Perganion Press New York 1978 pp 568-570
Palfreyman M G, Schmidt C J, Sorensen S M, Dudley M W, Kehne J H, Moser P, Gittos M W, Carr A A (1993). Electrophysiological, biochemical and behavioral evidence for 5-HT2 and 5-HT3 mediated control of dopaminergic function. Psychopharmacology 112: S60-7
Schifinan, S., Bronstein, Sterfeld, M., Shalom, A., Lev-Lehman, E., Weizman, A., Reznik, I., et al. (2003) A highly significant association between COMT haplotype and schizophrenia. Am. J. Hum. Genet. 71: 1296-1302.
Williams, G. V., and Goldman-Rakic, P. S. (1995) Modulation of memory fields by dopamine D1 receptors in prefrontal cortex. Nature 376: 572-5.
Wood, P. L. and Altar, C. A. Dopamine release in vivo from nigrostriatal, mesolimbic, and neocortical dopamine neurons: Utility of 3-methoxytyramine measurements. Pharmacol. Rev. 40:163-187, 1988.

### NEW REFERENCES

Jordan S, Koprivica V, Dunn R, Tottori K, Kikuchi T, Altar C A (2004) In vivo effects of aripiprazole on cortical and striatal dopaminergic and serotonergic function. Eur J Pharmacol 483:45-53.
Jordan S, Koprivica V, Chen R, Tottori K, Kikuchi T, Altar C A (2002) The antipsychotic aripiprazole is a potent, partial agonist at the human 5-HT1A receptor. Eur J Pharmacol 441:137-140.
Hirose T, Uwahodo Y, Yamada S, Miwa T, Kikuchi T, Mori T, Burns K D, Altar C A, Nabeshima T (2004) Efficacy and favorable side effect profile of the antipsychotic aripiprazole determined in rats with apomorphie-induced stereotypy, catalepsy, and ptosis induction. J Psychopharmacol (In Press).
Morris R (1984) Developments of a water-maze procedure for studying spatial learning in the rat. Journal of Neurosci Methods 11:47-60.
Tottori K, Nakai M, Uwahodo Y, Miwa T, Yamada S, Oshiro Y, Kikuchi T, Altar C A (2002) Attenuation of scopolamine-induced and age-associated memory impairments by the sienna and 5-hydroxytryptamine(1A) receptor agonist OPC-14523 (1-[3-[4-(3-chlorophenyl)-1-piperazinyl]propyl]-5-methoxy-3,4-dihydro-2[1H ]-quinolinone monomethanesulfonate). J Pharmacol Exp Ther 301:249-257.

The citation of these and other references throughout the description of this invention is provided merely to clarify that description and is not intended as an admission that any such reference is “prior art” to the invention. All references cited in this specification are incorporated herein in their entirety and to the same extent as if each reference was individually incorporated by reference.

1. A compound of the Formula A 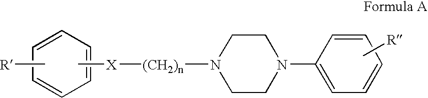
wherein R′ and R″ are independently selected for each position capable of substitution from the group consisting of halogen, hydroxyl, hydrogen, C₁-C₅ alkoxy, cyano (CN), and nitro (NO₂);
X is chosen from the group consisting of (C═O); O, NH, S, 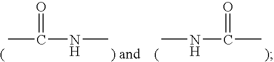
and
n is an integer from 1 to 6; and pharmaceutically acceptable salts thereof.

2. The compound according to claim 1 of the Formula B or the Formula C 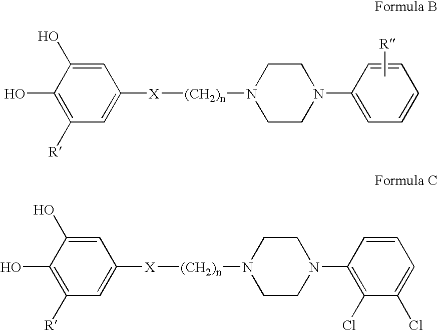
and pharmaceutically acceptable salts thereof.

3. (canceled)

4. The compound according to claim 1 wherein R′ is NO₂.

5. The compound according to claim 1 wherein X is chosen from the group consisting of

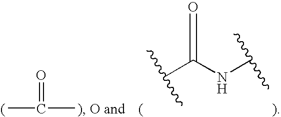

6. The compound according to claim 1 wherein n is 2, 3 or 4.

7. A compound of the Formula D 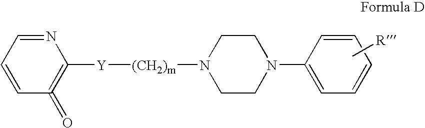
wherein R′″ is independently selected for each position capable of substitution from the group consisting of halogen, hydroxyl, hydrogen, C₁-C₅ alkoxy, cyano (CN) and nitro (NO₂);
Y is chosen from the group consisting of 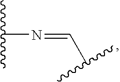
O, NH, S, 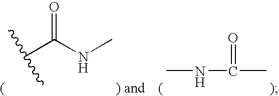
and
m is an integer from 1 to 6; and pharmaceutically acceptable salts thereof.

8. The compound according to claim 7 of the Formula E, the Formula F, the Formula G or the Formula H 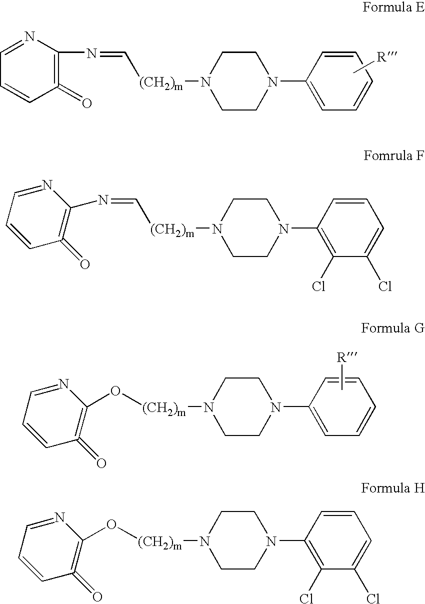
and pharmaceutically acceptable salts thereof.

9-11. (canceled)

12. The compound according to claim 2 having the Formula I, the Formula J, the Formula K, the Formula N or the Formula O 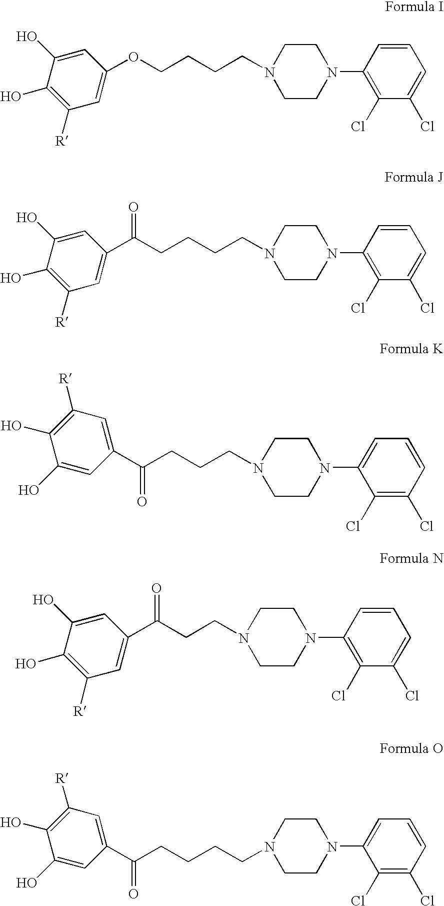
wherein R′ is selected from the group consisting of H, OH, CN and NO₂; and pharmaceutically acceptable salts thereof.

13-14. (canceled)

15. A compound having the Formula L or the Formula M 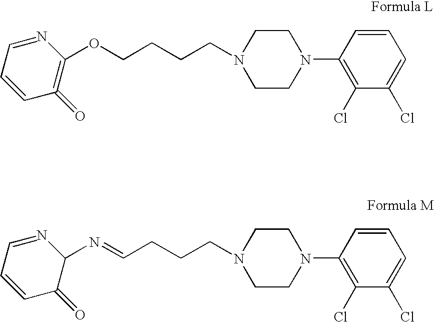
and pharmaceutically acceptable salts thereof.

16-18. (canceled)

19. The compound according to claim 2 having the Formula P or the Formula Q 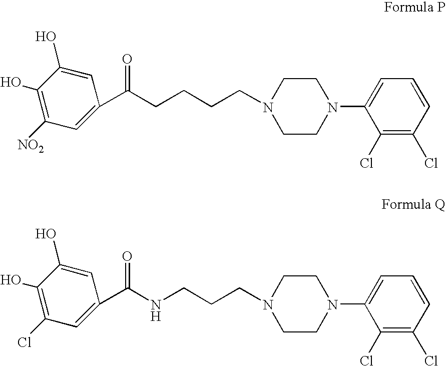
and pharmaceutically acceptable salts thereof.

20. (canceled)

21. A compound having the Formula R 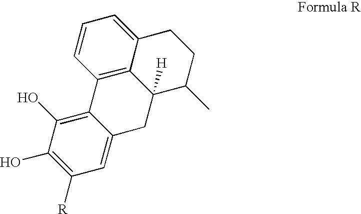
wherein R is chosen from the group consisting of OH, CN and NO₂ and pharmaceutically acceptable salts thereof.

22. A compound of the Formula S 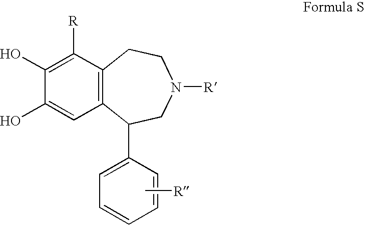
wherein R is chosen from the group consisting of OH, CN and NO₂;
R′ is chosen from the group consisting of H, C₁₋₆ alkyl and C₂₋₆ alkenyl;
R″ is chosen independently for each position capable of substitution from the group consisting of H, C₁₋₆ alkyl, halogen, hydroxyl, nitro and cyano; and enantiomers and diastereomers thereof and pharmaceutically acceptable salts thereof.

23. The compound according to claim 22 wherein R′ is chosen from the group consisting of H, CH₃ and

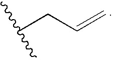

24. The compound according to claim 22 wherein R″ is chosen from the group consisting of H and CH₃.

25. A compound of the Formula T, the Formula U or the Formula V 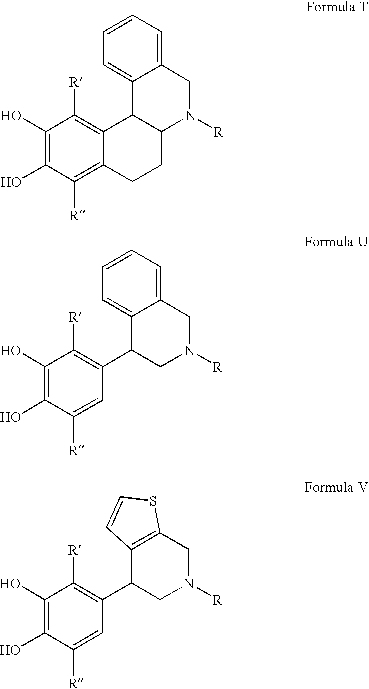
wherein R is selected from the group consisting of H and C₁₋₆ alkyl;
R′ and R″ are each independently selected from the group consisting of H, OH, CN and NO₂ with the proviso that, when R′ is the same as R″, R′ and R″ are not H,
and enantiomers, diastereomers and pharmaceutically acceptable salts thereof.

26-27. (canceled)

28. A compound of the Formula W 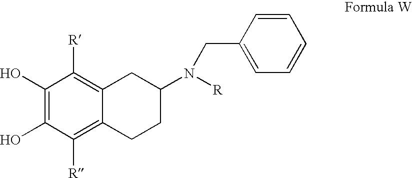
wherein R is selected from the group consisting of H and C₁-C₆ alkyl;
R′ and R″ are each independently selected from the group consisting of H, OH, CN and NO₂ with the proviso that when R′ is the same as R″, R′ and R″ are not H, and enantiomers and diastereomers thereof and pharmaceutically acceptable salts thereof.

29. A compound of the Formula X 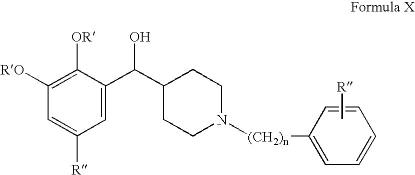
wherein R is selected from the group consisting of OH, NO₂ and CN;
R′ is selected from the group consisting of H and C₁₋₃ alkyl;
R″ is selected independently for each position capable of substitution from the group consisting of halogen, hydroxyl, hydrogen, C₁₋₅ alkyl, cyano and nitro; n is 0 to 6 and enantiomers and diasteroemers thereof and pharmaceutically acceptable salts thereof.

30. The compound according to claim 29 having the Formula Y 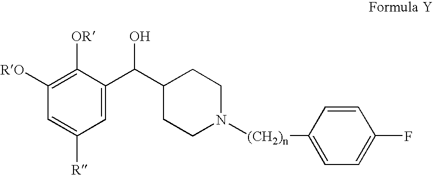
and pharmaceutically acceptable salts thereof.

31. The compound according to claim 29 wherein n is 2.

32. The compound according to claim 29 wherein R′ is H.

33. A pharmaceutical composition comprising at least one compound according to claim 1, claim 7, claim 15, claim 21, claim 22, claim 25, claim 28 or claim 29 in a therapeutically effective amount and in combination with one or more pharmaceutically acceptable carriers or diluents.

34. A method of treatment of Parkinson's Disease, Tourette's syndrome, cognitive impairment, depression, Alzheimer's disease, senile dementia, anxiety disorders, ischemic disease states, obsessive compulsive disorder, migraine, amyotrophic lateral sclerosis, epilepsy, eating disorders, premenstrual syndrome, attention deficit hyperactivity disorders, bipolar disorders, sexual dysfunction, or psychoses, comprising administering to a patient in need of such treatment a pharmaceutical composition according to claim 33.

35. A method for the treatment of schizophrenia comprising administering to a patient in need of such treatment a pharmaceutical composition according to claim 33.

36. The method of claim 35, wherein such treatment comprises treating the positive symptoms of schizophrenia.

37. The method of claim 35, wherein such treatment comprises treating the negative symptoms of schizophrenia.

38. The compound according to claim 12 having the Formula I, wherein R′ is NO₂.

39. The compound according to claim 22 having the Formula S, wherein R′ and R″ are both H, or wherein R′ is 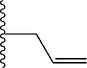
and R″ is H.

40. The compound according to claim 22 having the formula: 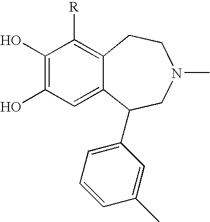
and pharmaceutically acceptable salts thereof.

41. The compound of claim 25 having the Formula T or the Formula U or the Formula V, wherein R′ is H.

42. The compound of claim 25 having the Formula T or the Formula U or the Formula V, wherein R″ is H.

43. The compound of claim 28 having the Formula W, wherein R′ is H or wherein R″ is H.

44. The compound according to claim 2, having one of the formulas: 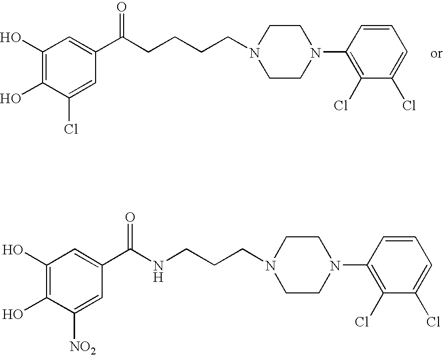
and pharmaceutically acceptable salts thereof.
